(19) United States
(12) Patent Application Publication
(10) Pub. No.: US 2012/0231086 A1
(43) Pub. Date: Sep. 13, 2012

(54) PROTEIN MATRIX VACCINES OF IMPROVED IMMUNOGENICITY

(76) Inventors: Kevin P. Killen, Needham, MA (US); Thomas J. Griffin, IV, Pembroke, MA (US); Ann Thanawastien, Pembroke, MA (US)

(21) Appl. No.: 13/394,147

(22) PCT Filed: Sep. 9, 2010
(86) PCT No.: PCT/US10/48311

§ 371 Date: May 17, 2012

Related U.S. Application Data
(60) Provisional application No. 61/276,183, filed on Sep. 9, 2009.

(51) Int. Cl. A61K 47/42; A61P 31/00; A61K 39/112; A61K 39/02; A61K 9/14; A61K 39/09

(52) U.S. Cl. 424/499; 424/278.1; 424/244.1; 424/258.1; 424/234.1; 424/282.1

(57) ABSTRACT

The present invention relates to immunogenic compositions containing an antigen of interest entrapped with a crosslinked carrier protein matrix, methods of making such vaccines, and methods of vaccine administration, wherein the immunogenicity of the protein matrix, and hence its effectiveness as a vaccine, is improved by controlling or selecting the particle size of the protein matrix particles to eliminate low molecular weight particles, e.g., less than 100 nm in diameter.

### CROSS-REFERENCE TO RELATED APPLICATION

This application claims priority to U.S. Provisional Application No. 61/276,183 filed Sep. 9, 2009, the contents of which are incorporated herein.

### FIELD OF THE INVENTION

The invention relates to immunogenic compositions, methods of making vaccines, and methods of vaccine administration. Specifically, the invention relates to protein capsular matrix vaccines featuring an antigen of interest entrapped in a crosslinked carrier protein matrix, wherein the particle size of the protein capsule matrix is controlled to increase immunogenicity of the composition. More specifically, the invention relates to matrix vaccine preparations in which low molecular weight matrix particles (e.g., <100 nm diameter) are eliminated. Advantages of increased immunogenicity are obtained in matrix vaccine formulations prepared to have a mean particle size diameter of greater than 100 nm diameter, that is, particle sizes of 150 nm, 200 nm, 500 nm, 1 micron, 2 microns or even larger are contemplated.

Many antigens, particularly those associated with a pathogen's capsule layer stimulate little or no immune response and complicate efforts to create effective vaccines against those antigens. Capsules are surface components of microbes that are typically composed of polymers of organic compounds such as carbohydrates, amino acids, or alcohols. Capsules are quite diverse chemically. The monomeric units that make up capsules (e.g., carbohydrates) can be linked together in various molecular configurations and can be further substituted with phosphate, nitrogen, sulfate, and other chemical modifications. The low immunogenicity of intact microbial capsules allow microbes to escape from the effector cells of host immune systems. Capsules can also be virulence factors which prevent microbes from being phagocytosed and killed by host macrophages and polymorphonuclear leukocytes.

Antibodies against capsules provide a potent defense against encapsulated organisms by fixing complement to the microbial surface, which can result in their lysis or their opsonization, uptake, and killing by phagocytic host immune cells. The most potent antibodies against microbial capsules are IgG antibodies. Capsular antigens are generally classified as T-independent antigens as they elicit immune responses that do not require T cell help and do not usually elicit long-lasting immunological memory responses. Covalent coupling of a protein to a capsular antigen renders the capsular antigen “T-dependent”, and such T-dependent antigens elicit a helper T cell-mediated (Tₕ-dependent) IgG response.

Various methods for rendering antigens more immunogenic and ideally T-dependent have been studied. Isolation of fragments of microbial surface polysaccharides often provides an immunogenic antigen capable of eliciting an immune response that will recognize the naturally occurring antigen in the microbial capsule. It has also been demonstrated that covalently linking an antigen to a carrier protein to provide a multivalent immunogen can greatly increase immunogenicity of the antigen and also promote the desired T-dependent immune response (or immune memory) that leads to protection of the host against subsequent infections by the antigen-bearing microorganism. For example, an unconjugated pneumoccoal vaccine, such as Merck's Pneumovax®, is efficacious against invasive pneumococcal disease in individuals, however it is often ineffective (e.g., in infants) at eliciting immunological memory and the desired protective immunity that would allow lifelong immunity and avoidance of constant re-immunization. Conjugate vaccines such as Pfizer's Prevnar®, having multiple pneumococcal polysaccharide antigens bound to a protein “carrier”, have been shown to be highly immunogenic even in 2-month old infants and to induce T-dependent immunity.

However, while conjugate vaccines are promising immunologically, they can be extremely difficult and complicated (and expensive) to manufacture, greatly deterring their distribution to all the patients and patient populations throughout the world that have need of them. For example, in the case of Prevnar®, each S. pneumoniae strain used to provide the 7 polysaccharide antigens used for conjugation is grown in a bioreactor; the cells are harvested; polysaccharide is extracted, purified, hydrolyzed to the appropriate size; individual antigens are then conjugated to the protein carrier; the conjugate is re-purified, mixed with the additional 6 other polysaccharide-protein complexes (conjugates) that were prepared in a similar manner; and the multi-conjugate mixture is finally adjuvanted with alum. It is estimated that there are more than 200 GMP steps in the manufacture of the heptavalent Prevnar® vaccine.

Recently, protein matrix vaccines have been proposed as an alternative to conjugate vaccines. See, US Patent Publn. 2008-0095803, incorporated herein by reference. Rather than covalently conjugating an antigen of interest to a carrier, a protein matrix vaccine entraps the antigen in a carrier protein matrix, prepared by crosslinking the carrier protein in the presence of the desired antigen. Significant covalent linking of the antigen to the carrier protein is avoided; rather, the antigen remains associated with the matrix by becoming entrapped by the protein carrier during matrix formation (crosslinking reaction). Such protein matrix vaccines have been shown to have much greater immunogenicity than vaccines prepared using the antigen alone; and protein matrix vaccines may also achieve an immunogenicity (and induction of T-dependent immunity) of the sort seen with conjugate vaccines. And such advantages are achieved with many fewer processing steps (e.g., half the steps) and complicated conjugation reactions necessary to produce conjugate vaccines.

Although protein matrix vaccines provide several advantages, the titer of antigen-specific antibodies elicited by protein matrix vaccines is often significantly lower than the titer elicited by a corresponding conjugate vaccine, if one is available. Thus, it is a persistent technical problem in the field to provide a means for increasing the immunogenicity of protein matrix vaccines, in order to exploit the scientific promise and manufacturing and cost advantages of this emerging technology. There is a continuing need for improved protein matrix vaccines having enhanced immunogenicity or potency.

### SUMMARY OF THE INVENTION

The present invention relates to an immunogenic composition comprising (1) an antigen of interest and (2) a carrier protein, wherein said carrier protein is crosslinked to form a protein matrix, said antigen of interest is entrapped by said protein matrix, and said composition is comprised of high molecular weight protein matrix particles, e.g., having a mean particle size greater than 100 nm diameter. Such compositions may be readily prepared by admixing the antigen and carrier protein components, initiating a crosslinking reaction to cause crosslinking of the carrier protein, followed by processing of the reaction product to eliminate lower molecular weight species (e.g., <100 nm diameter species). The protein matrix vaccine compositions of high molecular weight protein matrix particles according to the present invention have increased immunogenicity compared to compositions of low molecular weight protein matrix particles or compositions having a broad range of particle sizes including lower molecular weight protein matrix particles.

The present invention also provides a means of improving the immunogenicity of a protein matrix vaccine composition comprising the step of selecting the protein matrix particle sizes of the composition to eliminate lower molecular weight particles (less than 100 nm diameter) or selecting the protein matrix particle sizes of the composition to include particle sizes greater than 100 nn diameter. Preferred compositions according to the invention will have a particle size range from 120-2000 nm diameter or will include predominantly particles selected from within that range. Suitable compositions may be prepared directly after formation of the antigen-containing protein matrix by size fractionation of the crosslinking reaction mixture and selection of desired fractions comprised of high molecular weight species.

One embodiment of the invention is a vaccine composition containing an antigen of interest and a carrier protein matrix, where the antigen is entrapped with the carrier protein matrix to form a complex. In desirable embodiments of the invention, the antigen/protein complex has a mean particle size diameter above 100 nm. In more desirable embodiments of the invention, the complex has a mean particle size diameter of greater than 120 nm, greater than 170 nm, greater than 200 nm, greater than 500 nm, greater than 1000 nm, greater than 2000 nm or even larger, e.g., to the limits of the methodology for collecting the protein matrix particles. In yet more desirable embodiments of the invention, the protein matrix/antigen complexes of the vaccine composition will encompass a range of particle sizes above 100 nm in diameter, such as 100-2000 nm diameter, or selections within that range, e.g., 120-200 nm, 200-400 nm, 250-500 nm, 120-1000 nm, 200-2000 nm, and other such particle size ranges. In yet further desirable embodiments of the invention, the composition includes complexes having particle sizes of 170-185 nm diameter. It is demonstrated herein that raising the average complex particle size, or eliminating lower particle size components from the vaccine composition, leads to a surprising increase in immunogenicity with respect to the entrapped antigen. Moreover, larger protein matrix particles containing very small amounts of antigen are able to elicit immune responses surpassing or comparable to compositions of the antigen alone (uncomplexed) containing many times (e.g., 67-fold) more capsular antigen than the particle size-selected protein capsular matrix composition.

In desirable embodiments of the invention, the improved protein matrix vaccine compositions of the invention, when administered to a mammal, elicit a T cell-dependent immune response in the mammal (i.e., produce immunological memory in the vaccinated host).

In additional desirable embodiments, the vaccine composition further includes a two or more antigens of interest, for example, 2, 3, 4, 5, 6, 7, 8, 9, and/or 10 or more antigens of interest

Another aspect of the invention features a method of making a vaccine composition. This method involves (i) mixing an antigen of interest with a carrier protein to form a mixture of the antigen and the carrier protein, (ii) entrapping the antigen of interest with the carrier protein to form an antigen/protein complex, and (iii) selecting for complexes having a mean particle size diameter of greater than 100 nm.

In preferred embodiments of the invention, the antigen/protein complex has a mean particle size diameter of greater than 100 nm. More preferably, the antigen/protein matrix complex has a mean particle size diameter of greater than 500 nm. In yet further preferred embodiments, the antigen/protein complex has a mean particle size diameter of greater than 1000 nm. In still further preferred embodiments, the antigen/protein complex has a mean particle size diameter of greater than 1500 nm. In still further preferred embodiments, the antigen/protein complex has a mean particle size diameter of greater than 2000 nm.

In desirable embodiments of the invention, the immunogenic composition comprises an antigen of interest entrapped in a carrier protein matrix and further includes a pharmaceutically acceptable excipient.

In preferred embodiments, the invention features another method of making a vaccine composition. This method involves (i) mixing an antigen of interest with a carrier protein and (ii) adding a crosslinking agent capable of forming crosslinks between carrier protein molecules or between different sites of the same carrier protein molecule, (iii) initiating a crosslinking reaction between the carrier protein and the crosslinking agent, and (iv) selecting from the reaction product complexes having a particle size diameter of greater than 100 nm. In certain cases where the reactive groups of the crosslinking reagent and the reactive sites of the carrier protein will react on contact, the admixture and initiation steps (ii) and (iii) will occur simultaneously or may be considered one step. Additionally, it may be advantageous to quench the crosslinking reaction by including a step prior to step (iv) of attenuating the crosslinking reaction, e.g., by addition of an appropriate quenching or blocking agent.

Other features and advantages of the invention will be apparent from the following detailed description, the drawings, and the claims.

### BRIEF DESCRIPTION OF THE DRAWINGS

FIG. 1 is a graph showing a chromatogram of the size fractionation of a protein capasular matrix vaccine (PCMV) composition including Streptococcus pneumoniae type 14 polysaccharide antigen antigen entrapped in a crosslinked dominant negative mutant (DNI) of B. anthracis protective antigen (PPS 14:DNI PCMV) on a Sepharose® CL-2B column. The UV₂₈₀ absorbance is plotted, showing the amount of DNI eluting. Shading indicates fractions collected and pooled for later immunization experiments. The DNI mean particle size range for Pool 1 and the mean particle size for Pool 2 were determined by SEC-MALS-RI (Size Exclusion Chromatography with a Multi-Angle Laser Light detector and a Refractive Index detector).

FIG. 2 is an image showing the compositions prepared in Example 1 subjected to SDS polyacryamide gel electrophoresis and Coomasie blue staining, which illustrates the degree of crosslinking of the DNI carrier protein of the PCMV fractions. Uncrosslinked DNI protein migrates at 83 kDa in the absence of crosslinking (Composition 5). Composition 1 (Pool 1, particle size 200-120 nm), Composition 2 (Pool 2, particle size 63 nm), and Composition 3 (unfractionated PCMV, with DNI crosslinked using 0.25% glutaraldehyde) all showed extensive crosslinking of the DNI carrier protein as evidenced by the shift of bands to higher molecular weight species. Composition 4 (unfractionated PCMV prepared with 0.05% glutaraldehyde crosslinking agent) also showed crosslinking of DNI carrier protein but demonstrated a wider variety of bands ranging from lower molecular weight species to higher molecular weight bands.

FIG. 3 shows the results of a DNI capture ELISA in which captured matrix compositions are probed with anti-DNI antibodies to confirm crosslinked DNI integrity and with anti-PPS 14 antibodies to confirm entrapment and presentation of the polysaccharide (PPS 14) antigen in the captured DNI matrix. The test formulations were allowed to bind to anti-DNI capture antibody immobilized on an ELISA plate. Unbound material was washed away, and anti-DNI antibodies (panel A) or anti-polysaccharide antibodies (panel B) were used to detect DNI matrix or polysaccharide within the DNI matrix, respectively. Panel A shows the detection of DNI captured by the DNI capture antibody to demonstrate the vaccine formulations that contain DNI are bound by the capture antibody. Panel B shows that polysaccharide antigen was associated with DNI protein matrix, as detected by anti-polysaccharide specific detection antibody.

FIG. 4 is a bar graph showing the anti-PPS 14 IgG immune response in mice immunized with the four PCMV preparations, as set forth in Example 1. Mice were immunized three times at biweekly intervals and serum from each mouse was collected 10-12 days post-immunization. Mice immunized with 5 μg by protein of Composition 1 (Pool 1, PPS 14:DNI PCMV having particles sizes from 120-200 nm in diameter) generated equivalent or better anti-PPS 14 IgG titers than mice immunized with Composition 2 (Pool 2, PPS 14:DNI PCMV having mean particle size diameter of 63 nm), although Composition 1 contained half the dose of polysaccharide antigen as was administered in Composition 2.

FIG. 5 is a bar graph showing the anti-PPS 14 IgG antibody titer from mice immunized with 2 μg of Composition 1 (Pool 1+alum, containing 0.95 μg PPS 14) compared with anti-PPS 14 IgG antibody titer from mice immunized using 2 μg PPS 14 antigen alone. Mice were immunized three times at biweekly intervals and serum from each mouse was collected 10-12 days after immunization. The data demonstrate that sera from mice immunized with Pool 1 PCMV containing 0.95 μg PS shows enhanced PPS 14-specific IgG responses over time compared to sera from mice immunized with 2 μg of PPS 14 antigen alone.

FIGS. 6A and 6B show bar graphs of anti-PPS 14 IgG reciprocal endpoint titers from day 38 (bleed 3) in the study of Example 1. FIG. 6A illustrates titers from mice immunized with 5 μg by protein of Composition 1 (Pool 1+alum; containing 2.4 μg PPS 14; particle size range of 120-200 nm), Composition 2 (Pool 2+alum; 5.0 μg PPS 14; mean particle size 63 nm), Composition 3 (whole PCMV, crosslinked using 0.25% glutaraldehyde), Composition 4 (whole PCMV crosslinked using 0.05% glutaraldehyde), and the antigen control (PPS 14 alone; 5 μg). FIG. 6B shows the day 38 (bleed 3) data from sera from mice immunized with 2 μg by protein of Composition 1 (Pool 1+alum, containing 0.95 μg PPS) or with 2 μg PPS 14 alone. The data demonstrate that the anti-PPS 14 IgG response is significantly higher in mice immunized with PCMV formulations with highly crosslinked carrier proteins (i.e., PCMV compositions or fractions using 0.25% glutaraldehyde in the formation reaction), as compared with the PCMV prepared with 0.05% glutaraldehyde, which leads to less highly crosslinked DNI carrier and smaller particle sizes, and as compared to mice immunized with polysaccharide antigen alone.

FIG. 7 is a chromatogram showing fractionation of a PCMV prepared using the Salmonella typhi polysaccharide antigen Vi and the DNI carrier protein. The UV₂₈₀ absorbance of the Vi:DNI reaction product eluting from the Sepharose® CL-2B crosslinked agarose gel column is plotted. Fractions collected are shown by the short vertical lines originating from the x-axis. The shaded areas indicate the fractions that were collected and combined to make up Pools 1-4. The particle size for each pool was determined by dynamic light scattering (DLS), which indicates the largest particle size contained in the mixture, and that particle size (nm) is listed above each pool. For Pool 1, the particle size was calculated at 179 nm in diameter. The particles in Pool 2 were 171 nm in diameter. The particles in Pool 3 were 198 nm in diameter, and the particles in Pool 4 were calculated to be 185 nm in diameter.

FIG. 8 is a bar graph showing the results of an assay measuring anti-Vi IgG immune responses in mice immunized with the four PCMV preparations prepared as described in Example 2: Pool 1+alum (particle size 179 nm), Pool 2+alum (particle size 171 nm), Pool 3+alum (particle size 198 nm), Pool 4+alum (particle size 185 nm), whole (unfractionated) PCMV (0.25% glutaraldehyde), and the antigen control preparation (10 μg Vi antigen alone).

FIG. 9 is a bar graph showing the anti-Vi IgG endpoint titers at Day 38 for mice immunized, respectively, with one of the PCMV preparations described in Example 2, compared to immunization with capsular antigen alone.

FIG. 10 is a chromatogram of the fractionation of a PCMV preparation using Streptococcus pneumoniae type 14 polysaccharide antigen and DNI carrier protein crosslinked using 0.25% glutaraldehyde. Fractions were separated on a Sepharose® CL-2B crosslinked agarose size exclusion column. The UV₂₈₀ absorbance of the protein is plotted. The fractions collected are shown by the short vertical lines originating from the x-axis. The shaded areas indicate the fractions that were collected and combined for Pools 1, 2, 3 and 4.

FIG. 11 is an image of a Coomassie blue-stained SDS polyacrylamide electrophoresis gel (4-12% Bis-Tris gel) demonstrating cross-linking integrity of the PPS 14:DNI PCMV compositions described in Example 3. The four PCMV pools and the whole (unfractionated) PCMV reaction mixture all showed extensive crosslinking of the DNI carrier protein as evidenced by the lack of migration into the stacking gel. The appearance of a smear below the well for Pool 4, similar to the smear below the well for the whole PCMV reaction indicates the presence of lower molecular weight species in these samples.

FIG. 12 shows the results of a DNI capture ELISA and probes with anti-PPS 14 or anti-DNI detection antibodies to confirm entrapment of polysaccharide antigen and DNI crosslinking integrity. Varying concentrations of PPS 14:DNI PCMV pools (Pools 1-4; see, Example 3 and FIG. 10), the whole PCMV reaction mixture, or crosslinked DNI with exogenously added PPS 14 polysaccharide were incubated with immobilized DNI capture antibody. FIG. 12A shows the detection of PPS 14 that remains associated with the crosslinked DNI protein matrix after capture and washing; FIG. 12B shows the detection of DNI captured by the immobilized DNI capture antibody to confirm particles captured in the ELISA are composed of DNI.

FIG. 13 is a bar graph showing the anti-PPS 14 IgG immune response in mice immunized with the PCMV preparations described in Example 3, compared with those of mice immunized with the antigen control PPS 14 alone, or Prevnar® conjugate vaccine. FIG. 13A shows PPS 14-specific IgG from mice immunized with PCMV containing 0.5 μg of DNI and varying amounts of entrapped antigen, i.e., 0.03 μg PPS 14 (Pool 1), 0.06 μg PPS 14 (Pool 2), 0.13 μg PPS 14 (Pool 3), and 0.48 μg PPS 14 (Pool 4), compared against whole PCMV, 0.5 μg PPS 14 alone, or Prevnar® (which contains 2 μg PPS 14). FIG. 13B shows PPS 14-specific IgG from mice immunized with PCMV containing 2 μg DNI and varying amounts of entrapped antigen, i.e., 0.12 μg PPS 14 (Pool 1), 0.22 μg PPS 14 (Pool 2), 0.52 μg PPS 14 (Pool 3), and 1.91 μg PPS 14 (Pool 4), compared against whole PCMV, 2 μg PPS 14 alone, or Prevnar® (which contains 2 μg PPS 14). Endpoint titer cutoff is calculated as the titer that is 2 standard deviations above the mean of the negative control (pre-immune sera). Pools 1 and 2, which contained larger sized DNI carrier particles, elicited comparable anti-PPS 14 responses to Prevnar® conjugate vaccine, at significantly less dosage of PPS 14 antigen.

FIG. 14 shows anti-PPS 14 IgG endpoint titers from individual sera collected from Bleed 3 (Day 39) upon completion of the 0.5 μg immunization regimen described in Example 3. Mice immunized with PCMV Pool 1 (0.5 μg DNI, 0.03 μg PPS 14) and Pool 2 (0.5 μg DNI, 0.06 μg PPS 14) developed comparable anti-PPS 14-specific IgG GMT compared to mice immunized with Prevnar®. Immunization with PCMV delivered a significantly reduced amount of PPS 14 antigen compared with the conjugate vaccine (Prevnar®), yet elicited significant anti-PPS 14-specific IgG responses, and comparable anti-PPS 14-specific responses in the cases of Pools 1 and 2, compared to the response induced by the 2 μg dose of PPS 14 contained in the Prevnar® formulation used.

FIG. 15 shows anti-PPS 14 IgG endpoint titers from individual sera collected from Bleed 3 (Day 39) upon completion of the 2 μg immunization regimen described in Example 3. Mice immunized with PCMV Pool 1 (0.5 μg DNI, 0.12 μg PPS 14) and Pool 2 (0.5 μg DNI, 0.22 μg PPS 14) developed comparable anti-PPS 14-specific IgG GMT compared to mice immunized with Prevnar® conjugate vaccine. Immunization with PCMV delivered a significantly reduced amount of PPS 14 antigen compared with the conjugate vaccine (Prevnar®), yet elicited significant anti-PPS 14-specific IgG responses, and comparable anti-PPS 14-specific responses in the cases of Pools 1 and 2, compared to the response induced by the 2 μg dose of PPS 14 contained in the Prevnar® formulation used. Similar to the results seen in FIG. 14, immunization with PCMV fractions including large particle size DNI matrices elicited, at a significantly reduced dosage of PPS 14, comparable anti-PPS 14-specific IgG responses to titers induced by the 2 μg dose of PPS 14 in Prevnar®.

FIG. 16 is a chromatogram of the fractionation of the Vi:DNI PCMV preparation described in Example 4 on a Sepharose® CL-2B crosslinked agarose gel column. The absorbance at UV₂₈₀ is plotted. Fractions collected are shown by the short vertical lines originating from the x-axis. The shaded areas indicate the fractions that were collected for Pools 1, 2, 3 and 4 (see, Example 4).

FIG. 17 shows the results of a DNI capture ELISA to confirm entrapment and association of the Vi antigen in the crosslinked DNI matrix. FIG. 17A shows detection of Vi that remains associated with DNI matrix protein by anti-Vi detection antibody. FIG. 17B shows detection of DNI captured by the DNI capture antibody.

FIG. 18 is an image of a Coomassie blue-stained SDS-PAGE gel (4-12% Bis-Tris gel) illustrating crosslinking integrity of the Vi:DNI PCMV fraction pools and the whole PCMV described in Example 4. The PCMV pooled fractions and the whole PCMV reaction mixture contained very high molecular weight species that did not visibly migrate into the gel and remained in the loading wells. Uncrosslinked DNI (control) showed a low molecular weight band after electrophoresis (unlabeled arrow)

FIG. 19 is a bar graph showing anti-Vi IgG immune responses in mice immunized with the PCMV and control preparations described in Example 4. Anti-Vi IgG endpoint titers from individual sera collected from Bleed 1 (day 8), Bleed 2 (day 22), and Bleed 3 (day 41) are shown. Vi-specific IgG responses are determined for mice immunized with PCMV formulations containing 10 μg of DNI with an undetermined dose of Vi for Pools 1-4 and whole PCMV. Control groups of mice were immunized with 5 μg of Vi contained in a Vi-BSA conjugate or with 10 μg Vi polysaccharide antigen alone derived either from Salmonella typhi or from Citrobacter freundii. Sera from mice immunized with the larger carrier matrix particles (Pools 1-3) developed higher Vi-specific IgG responses than sera from mice immunized with 10 μg Vi alone. Immunization with Pool 4 (smaller particles) or whole PCMV generated Vi-specific antibody responses similar to when mice were immunized with Vi alone.

FIG. 20 is a bar graph showing anti-Vi endpoint titers of the four PCMV and control preparations described in Example 4. Anti-Vi IgG endpoint titers were determined from individual sera collected from Bleed 3 (Day 41) upon completion of the immunization regimen.

FIG. 21 is a bar graph showing anti-PPS 14 IgG/IgM ratio. Data from the Day 10, 54, 239, 243, and 260 blood samples were collected and analyzed for PPS 14-specific IgG and IgM and the IgG/IgM ratio was calculated. The high and ascending IgG/IgM ratios over the course of the immunization observed for the Pool 1 and Pool 2 groups is an indication of an immunological “memory” response. The weakening response over time and low IgG/IgM ratios of the controls indicate that immune memory was not induced by the preparations containing polysaccharideantigen alone.

### DETAILED DESCRIPTION OF THE INVENTION

Protein matrix vaccines, and particularly protein capsular matrix vaccines (PCMVs), are described in international patent publication WO 2008/021076 (Mekalanos), published Feb. 21, 2008, and US patent publication no. US 2008-0095803 (Mekalanos), published Apr. 28, 2008, both incorporated herein in their entirety. These publications teach that protein matrix vaccines have the potent immunological properties of typical PS-protein conjugate vaccines but desirably differ from conjugate vaccines in that no significant covalent bonding is required to couple the antigen of interest to the carrier protein. Rather, the antigen of interest, e.g., polysaccharides, capsular organic polymers or other antigens, is entrapped within a carrier protein matrix.

When a capsular biopolymer or polysaccharide of a pathogen is entrapped in a crosslinked protein matrix, such vaccines are termed protein capsular matrix vaccines (PCMVs). As described in WO 2008/021076 and US 2008-0095803, PCMVs were produced including ones based on the model T-independent capsular antigen, poly-gamma-D-glutamic acid (PGA), as well as alginic acid (alginate) and dextran, and the exemplary carrier protein, Dominant Negative Inhibitor mutant (DNI). DNI is a mutated form of Protective Antigen (PA) of B. anthracis and was produced from Escherichia coli by the method of Benson, et al., Biochemistry, 37:3941-3948 (1998).

The present invention relates to discoveries and observations made in respect of enhancing the immunogenicity of protein matrix vaccine compositions.

In order that the invention may be more clearly understood, the following abbreviations and terms are used as defined below.

A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step. It is also understood that an element or step “selected from the group consisting of” refers to one or more of the elements or steps in the list that follows, including combinations of any two or more of the listed elements or steps.

The term “administering” as used herein in conjunction with a vaccine composition, means providing the vaccine composition to a subject such as a human subject in a dose sufficient to induce an immune response in the subject, where the immune response results in the production of antibodies that specifically bind an antigen contained in the vaccine composition (i.e., which antigen, in therapeutic vaccines, corresponds to an antigenic marker on a pathogen). Administering desirably includes intramuscular injection, intradermal injection, intravenous injection, intraperitoneal injection, subcutaneous or transcutaneous injection, inhalation, or ingestion, as appropriate to the dosage form and the nature and activity of the vaccine composition to be administered. Administering may involve a single administration of a vaccine or administering a vaccine in multiple doses. Desirably, a second (“booster”) administration is designed to boost production of antibodies in a subject to prevent infection by an infectious agent. The frequency and quantity of vaccine dosage depends on the specific activity of the vaccine and can be readily determined by routine experimentation.

The term “cross-link” or “crosslink” refers to the formation of a covalent bond between two molecules, macromolecules, or combination of molecules, e.g., carrier protein molecules, or between two sites of the same molecule, e.g., two amino acid residues of the same protein, either directly, when a “zero-length” linker is used (creating a direct bond), or by use of bifunctional crosslinker molecule that forms a molecular bridge or link between two reactive sites. Bifunctional crosslinkers exhibit two functional groups, each capable of forming a covalent bond with one of two separate molecules or between two separate groups in the same molecule (i.e., so as to form “loops” or “folds” within a molecule such as a carrier protein). Exemplary linkers include bifunctional crosslinkers which are capable of crosslinking two carrier proteins.

The term “antigen” as used herein refers to any molecule or combination of molecules that is specifically bound by an antibody or an antibody fragment.

The term “bifunctional crosslinker” or “bifunctional linker” as used herein means a compound that has two functional groups, each separately capable of forming a covalent bond with reactive groups on two separate molecules, atoms, or collections of molecules desired to be linked together. Exemplary bifunctional linkers are described, for example, by G. T. Hermanson, Bioconjugate Techniques (Academic Press, 1996) and Dick and Beurret, “Glycoconjugates of Bacterial Carbohydrate Antigens,” in Conjugate Vaccines (Cruse and Lewis, eds), Contrib. Microbiol. Immunol. Basel, Karger, 1989, vol. 10, pp. 48-114). Desirably a bifunctional linker is glutaraldehyde, bis[sulfosuccinimidyl]suberate, or dimethyl adipimidate.

The term “linker” or “crosslinker” as used herein refers to a compound capable of forming a covalent chemical bond or bridge that joins two or more molecules or two or more sites in the same molecule. Desirable linkers include, e.g., glutaraldehyde or other dialdehydes of the formula OHC—R—CHO, where R is a linear or branched divalent alkylene of 1 to 12 carbon atoms, a linear or branched divalent heteroalkyl of 1 to 12 atoms, a linear or branched divalent alkenylene of 2 to 12 carbon atoms, a linear or branched divalent alkynylene of 2 to 12 carbon atoms, a divalent aromatic radical of 5 to 10 carbon atoms, a cyclic system of 3 to 10 atoms, —(CH₂CH₂O)_{q}CH₂CH₂— in which q is 1 to 4, or a direct chemical bond linking two aldehyde groups. Linking may be direct without the use of a linking (bridging) molecule. For example, a carboxyl group, for instance on the side chain of an Asp or Glu residue in a carrier protein carboxyl group may be linked directly to a free amino group, for instance on the side chain of a Lys residue, using carbodiimide chemistry or enymatically using transglutamidases which catalyze crosslinking between free amino groups and carboxamide groups, e.g., of Gln.

The term “boost” in the context of eliciting production of antibodies refers to the activation of memory B-cells that occurs during a second exposure to an antigen. This is also referred to as a “booster response” and is indicative of a long-lived “secondary” memory immune response, resulting in the long-lived production of antibodies.

The term “carrier protein” in the context of a vaccine composition refers to a protein used in a vaccine composition that evokes an immune response to itself and/or to an antigen associated with or complexed with such carrier protein. In a protein matrix vaccine composition, an antigen is associated with a carrier protein that is crosslinked to form a protein matrix, thereby entrapping antigen to form a complex with the carrier protein, preferably without significant covalent linkage of antigen to the matrix. In a conjugate vaccine composition, an antigen is reacted with a carrier protein, so that the antigen and carrier protein are covalently linked to each other, by design. Desirably, the carrier protein contains an epitope recognized by a T cell. Also encompassed by the definition of a “carrier protein” are multi-antigenic peptides (MAPs), which are branched peptides. Desirably, a MAP includes lysine (Lys). Exemplary desirable carrier proteins include toxins and toxoids (chemical or genetic), which may be mutated, e.g., to reduce reactogenicity. Suitable carrier proteins include, e.g., diphtheria toxin or a mutant thereof, diphtheria toxoid, tetanus toxin or a mutant thereof, tetanus toxoid, Pseudomonas aeruginosa exotoxin A or a mutant thereof, cholera toxin B subunit, tetanus toxin fragment C, bacterial flagellin, pneumolysin, listeriolysin O (LLO, and related molecules), an outer membrane protein of Neisseria menningitidis, Pseudomonas aeruginosa Hcp1 protein, Escherichia coli heat labile enterotoxin, shiga-like toxin, human LTB protein, a protein extract from whole bacterial cells, the dominant negative inhibitor mutant (DNI) of the Protective Antigen of Bacillus anthracis, or Escherichia coli beta-galactosidase, or any other protein that can be cross-linked by a linker.

The term “entrapped” as used herein in reference to an antigen means association or complexing of an antigen with a carrier protein, in particular a carrier protein crosslinked to form a matrix which forms the association or complex with the antigen, such that antigen remains in the complex with carrier protein under physiological conditions. Desirably, the antigen is entrapped in a complex with carrier proteins in the absence of significant covalent bonding between the antigen and a carrier protein. Absence of significant covalent bonding, as used herein, refers to no more than 50% of the antigen being covalently bound to a carrier protein. Desirably, no more than 40%, no more than 30%, no more than 20%, no more than 10%, or desirably, no more than 5% of the antigen is covalently bonded to carrier protein in a protein matrix vaccine composition.

By “infection” is meant the invasion of a subject by a microbe, e.g., a bacterium, fungus, parasite, or virus. The infection may include, for example, the excessive multiplication of microbes that are normally present in or on the body of a subject or multiplication of microbes that are not normally present in or on a subject. A subject is suffering from a microbial infection when an undesirably (e.g., pathogenic) excessive microbial population is present in or on the subject's body or when the presence of a microbial population(s) is damaging the cells or causing pathological symptoms in a tissue of the subject.

By “infectious agent” is meant a microbe that causes an infection.

The term “immunogenic” refers to a compound that induces an immune response in a subject. Desirably, an immune response is a T cell-dependent immune response that involves the production of IgG antibodies.

The term “microbial capsular polymer” refers to a polymer present in or on the capsule coating of a microbe. Desirably, a microbial capsular polymer is an organic polymer such as a polysaccharide, phosphopolysaccharide, polysaccharide with an amino sugar with a N-acetyl substitution, polysaccharide containing a sulfonylated sugar, another sulfate-modified sugar, or phosphate-modified sugar, polyalcohol, polyamino acid, teichoic acid, or an O side chain of a lipopolysaccharide.

“Monomer” refers to a molecular structure capable of forming two or more bonds with like monomers, often yielding a chain or a series of branched, connected chains of repeating monomer substructures, when part of a “polymer.”

“Organic polymer” refers to a polymer composed of covalently linked monomers each composed of carbon, oxygen, hydrogen, or nitrogen atoms or phosphate or sulfate moieties. Desirably, an organic polymer is a polysaccharide, phosphopolysaccharide, polysaccharide with an amino sugar with a N-acetyl substitution, polysaccharide containing a sulfonylated sugar, another sulfate-modified sugar, or phosphate-modified sugar, sugar, polyalcohol, polyamino acid, teichoic acid, and an O side chain of lipopolysaccharide.

“Polyalcohol” means a hydrogenated form of a carbohydrate where a carbonyl group has been reduced to a primary or secondary hydroxyl group. Exemplary polyalcohols are a polyalkylene oxide (PAO), such as a polyalkylene glycols (PAG), including polymethylene glycol, polyethylene glycol (PEG), methoxypolyethylene glycol (MPEG) and polypropylene glycol; poly-vinyl alcohol (PVA); polyethylene-co-maleic acid anhydride; polystyrene-co-malic acid anhydride; dektrans including carboxymethyl-dextrans; celluloses, including methylcellulose, carboxymethylcellulose, ethylcellulose, hydroxyethylcellulose carboxyethylcellulose, and hydroxypropylcellulose; hydrolysates of chitosan; starches such as hydroxyethyl-starches and hydroxy propyl-starches; glycogen; agaroses and derivates thereof; guar gum; pullulan; insulin; xanthan gum; carrageenan; pectin; alginic acid hydrolysates; sorbitol; an alcohol of glucose, mannose, galactose, arabinose, gulose, xylose, threose, sorbose, fructose, glycerol, maltose cellobiose, sucrose, amylose, amylopectin; or mono propylene glycol (MPG).

“Poly amino acid” or “polyamino acid” means at least two amino acids linked by a peptide bond. Desirably, a poly amino acid is a peptide containing a repetitive amino acid sequence or a chain of the same amino acid (i.e., a homopolymer).

The term “reducing a Schiff base” refers to exposing azomethine or a compound of the formula R₁R₂C═N—R₃ (where R₁, R₂, and R₃ are chemical substructures, typically containing carbon atoms) to a reducing agent that saturates the double bond of the Schiff base with hydrogen atoms. Methods of reducing are known to those skilled in the art.

The term “specifically binds” as used herein in reference to an antibody or a fragment thereof, means an increased affinity of an antibody or antibody fragment for a particular antigen, e.g., a protein or segment thereof, relative to an equal amount of any other antigen. An antibody or antibody fragment desirably has an affinity for its antigen that is least 2-fold, 5-fold, 10-fold, 30-fold, or 100-fold greater than for an equal amount of any other antigen, including related antigens, as determined using standard methods such as an enzyme linked immunosorbent assay (ELISA).

By “subject” is meant an animal that can be infected by a microbe. Desirably, a subject is a mammal such as a human, monkey, dog, cat, mouse, rat, cow, sheep, goat, or horse. A human subject may be an adult human, child, infant, toddler, or pre-pubescent child.

A “T cell-independent antigen” refers to an antigen which results in the generation of antibodies without the cooperation of T lymphocytes. The T cell-independent antigen may directly stimulates B lymphocytes without the cooperation of T lymphocytes. Exemplary desirable T cell-independent antigens include capsular antigen poly-gamma-D-glutamic acid (PGA), alginic acid (algenate), dextran, polysaccharides (PS), poly amino acids, polyalcohols, and nucleic acids.

Protein matrix vaccine compositions of the present invention do not require covalent linkage between the antigen intended to evoke an immune response and the carrier protein used to form the matrix. This advantageously simplifies the preparation of protein matrix vaccine compositions, reducing the cost of their preparation compared to conjugate vaccine technology. Polysaccharide (PS)-protein conjugate vaccines have proved to be prohibitively expensive to produce and sell in the developing world. Conventional conjugate vaccines are difficult to produce cheaply because of the highly specialized chemistry required for each vaccine and the costs of production and purification of both PS antigen and carrier protein.

Vaccine compositions according to the present invention address a need for vaccines that can safely induce immunity against previously intractable antigens. Vaccine compositions as described herein may be monovalent (having a single antigen to induce an immune response) or multivalent (having multiple antigens to induce a multiplex immune responses). Vaccine compositions containing Toll-like receptor (TLR) ligands have been shown to evoke immune responses for otherwise intractable antigens, but they tend to be unsafe because TLR ligands are often proinflammatory, toxic in even small doses, reactogenic, and likely to cause adverse symptoms compared to compositions of this invention.

The meaning of other terms will be understood by the context in which they appear or as understood by skilled practitioners in the art, including practitioners in the fields of organic chemistry, pharmacology, microbiology, protein biochemistry, and immunology.

The present invention relates to an immunogenic composition comprising (1) an antigen of interest and (2) at least one carrier protein, wherein said carrier protein is crosslinked to form a protein matrix, said antigen of interest is entrapped by said protein matrix, and said composition is comprised of high molecular weight protein matrix particles, e.g., having a mean particle size greater than 100 nm diameter, desirably a mean particle size in the range of 100-2000 nm diameter or larger. Such compositions may be readily prepared by admixing the antigen and carrier protein components, initiating a crosslinking reaction to cause crosslinking of the carrier protein, followed by processing of the reaction product to eliminate lower molecular weight species (e.g., <100 nm diameter species). It has been discovered that producing protein matrix vaccine compositions having large protein matrix particle size, e.g., >100 nm in diameter, lead to increased immunogenicity of the carried (entrapped) antigen. Moreover the improvement in immunogenicity by increasing protein matrix particle size becomes more pronounced with increasing particle size, such that particles greater than 200 nm in diameter, 300 nm in diameter, 500 nm in diameter, 750 nm in diameter, or 1000 nm (1 μm) in diameter or even larger are contemplated herein. The protein matrix vaccine compositions of high molecular weight protein matrix particles according to the present invention have increased immunogenicity compared to compositions of low molecular weight protein matrix particles or compositions having a broad range of particle sizes including lower molecular weight protein matrix particles.

The present invention features, in particular, protein capsular matrix vaccine compositions of high molecular weight protein capsular matrix particles and methods of making and administering such compositions to provide immunity against antigens, particularly T cell-independent antigens or antigens which normally evoke weak immune responses, such as, e.g., polysaccharides (PS), polyalcohols, poly amino acids, and other organic polymers. The vaccine compositions of the invention have the potent immunological properties of typical PS-protein conjugate vaccines but desirably differ from conjugate vaccines in that no significant covalent atomic bonding is required to couple the antigen of interest, e.g., PS or capsular organic polymer, to the carrier protein. Rather, the antigen of interest, e.g., PS or capsular organic polymers, is entrapped with the carrier protein matrix. For example, a protein matrix may be formed by covalent cross-linking carrier protein molecules to themselves in the presence of soluble antigen, e.g., PS or capsular organic polymers. Carrier proteins that are highly crosslinked to each other can form a matrix that can capture an antigen and facilitate the uptake of that antigen and the stimulation of antibody production in immune cells. As demonstrated herein, the immunogenicity of a protein capsular matrix vaccine composition is further enhanced by selecting the protein matrix particle sizes of the composition to eliminate lower molecular weight particles (less than 100 nm diameter) or selecting the protein matrix particle sizes of the composition to include particle sizes greater than 100 nm in diameter.

The carrier protein matrix may be in the form of a “mesh” that encloses the antigen or a series of “beads on a string” where the antigen is the “string”, the protein or complexes of cross-linked proteins is the “bead” in this analogy. The antigen is entrapped with the carrier protein if the carrier protein encircles the antigen to form a ring around the antigen or a 3-dimensional mesh in which the antigen is tangled within.

In desirable embodiments, molecules of the carrier protein are covalently crosslinked, for example, the covalent linkage contains a peptide bond between a primary amino group of a lysine side chain and a carboxy group of an aspartate or glutamate side chain. In other desirable embodiments, covalent crosslinks can be initiated using crosslinkers such as compounds of the formula OHC—R—CHO, where R is a linear or branched divalent alkylene of 1 to 12 carbon atoms, a linear or branched divalent heteroalkyl of 1 to 12 atoms, a linear or branched divalent alkenylene of 2 to 12 carbon atoms, a linear or branched divalent alkynylene of 2 to 12 carbon atoms, a divalent aromatic radical of 5 to 10 carbon atoms, a cyclic system of 3 to 10 atoms, —(CH₂CH₂O)_{q}CH₂CH₂— in which q is 1 to 4, or a direct chemical bond linking two aldehyde groups. In preferred embodiments, the covalent linkage is formed using glutaraldehyde as a crosslinking agent, or alternatively such agents as m-maleimidobenzoyl-N-hydroxysuccinimide ester, carbodiimide, or bis-biazotized benzidine, bis[sulfosuccinimidyl]suberate, or dimethyl adipimidate may be used. Although not required in the formation of a protein matrix vaccine composition, the antigen of interest may by covalently bound to the carrier protein, for example, to an extent that is incidental to the formation of the crosslinked carrier protein matrix, e.g., due to unblocked reactive groups or terminal amino or carboxyl groups or hydroxyl groups that may exist on the antigen. In general, covalent linkage of antigen to carrier is not an object in the formation of protein matrix vaccines. For the purposes of the invention, protein matrix vaccines are vaccine compositions wherein no more than 50% of the antigen is covalently linked to carrier protein.

In desirable embodiments, the antigen and the carrier protein are non-covalently linked. Such non-covalent linkage may involve a hydrophobic interaction, ionic interaction, van der Waals interaction, or hydrogen bond. Non-covalent linkage can include physical geometric configurations that non-covalently associate antigen with protein complexes (i.e., as in the “bead on a string” analogy above).

Vaccine compositions of the invention may be prepared using any of many possible linkers to crosslink any of many possible carrier proteins in the presence of any antigen of interest. Exemplary and preferred linkers, carrier proteins, and antigens of interest are discussed herein.

Polysaccharides (PS) are polymers of saccharides (sugars). PS derived from microbial capsules are the primary antigenic components involved in protective immunity against encapsulated bacterial pathogens such as Neisseria meningitidis, Streptococcus pneumoniae, Salmonella typhi, and Haemophilus influenzae Type B. Immunization of adolescents and adults with vaccines based on microbial polysaccharides has been successful in reducing disease burden, but has proven less effective in providing protective immunity to infants and young children (i.e., children less than 24 months of age). Young children have not yet developed a mature adaptive immune repertoire and T cell-independent antigens such as capsular PS are poorly immunogenic and do not lead to long-term protective immune responses (i.e., an immunological memory response) in such young vaccine recipients.

A T cell-independent antigen such as polysaccharide can be converted to a T cell-dependent antigen by chemical coupling of polysaccharide to protein. This process, known as “conjugation”, involves the formation of covalent bonds between atoms in the polysaccharide structure and side chain atoms of amino acids present in the “carrier” protein. Such “conjugate vaccines” more efficiently promote the induction of B-cell maturation and isotype switching, leading to much higher levels of antibody with the correct anti-PS protective profile. Protective antibodies have high affinity for their polysaccharide antigens, and typically are of the Immunoglobulin G (IgG) subclass, a long-lived antibody with complement fixing and opsonization effector activity.

A T cell-independent antigen generally does not stimulate lasting immunity, i.e., the production of IgG antibodies, but may stimulate the production of less potent and more temporary IgM antibodies. As such, polysaccharide antigens alone do not typically produce booster responses of IgG. However, polysaccharides do produce booster responses if primary immunization is performed with a PS-protein conjugate because memory cells induced by the conjugate have already been programmed to produce IgG. Indeed, the booster response in vaccinated animals or humans is thought to mimic the protective response due to exposure to a microbe displaying the PS; this long term memory is critical for a vaccine to work in providing protective immunity to immunized subjects years after their immunization. Thus, PS-protein conjugates are valued for (1) their ability to induce high levels of IgG against PS antigens, and (2) their ability to induce memory immune responses against PS antigens. Polysaccharide antigens typically do not display these properties and thus are inferior antigens. The difficulty in synthesizing conjugate vaccines and their cost of production has slowed the development of conjugate vaccines for many bacterial diseases where an immune response to a polysaccharide antigen may be protective.

Other T cell-independent antigens include homopolymers of amino acids, such as poly-gamma-D-glutamic acid (PGA), and polyalcohols. Most biopolymers are T cell-independent antigens. Polymers can crosslink Immunoglobulin (Ig) receptors on B-cells that recognize them due to the repetitive nature of their chemical structures (and thus epitopes). Thus polymers can activate B-cells for production of anti-polymer IgM in the same way that polysaccharides do. For example, an amino acid homopolymer, poly-gamma-D-glutamic acid (PGA) of Bacillus anthracis, is a capsular polymer that is poorly immunogenic and also a T cell-independent antigen. Vaccines composed of PGA conjugated to protein carriers are highly immunogenic, able to induce anti-PGA IgG, and immunological memory to PGA. Hence, most polymers respond like PS in terms of their immunogenicity because they cannot be processed and displayed in the context of MHC-II and thus cannot recruit T cell help. An exception is found in some naturally-occurring polymers that interact with another class of receptor termed Toll-like receptors (TLRs). Once activated, TLRs can induce production of cytokines by host cells and produce changes in the adaptive immune response. Some PS are covalently attached to TLR ligands or contaminated with such ligands. For example, lipopolysaccharides (LPS) are PS that are highly immunogenic and induce IgG and memory responses; the lipid A moiety of LPS is a TLR ligand and may be responsible for the immunological properties.

Conventional conjugate vaccines are difficult to produce cheaply because costs of production and purification of both PS antigen and carrier protein and the specific chemistry involved in each polysaccharide-protein conjugation. Usually both need to be quite pure before conjugation chemistry can be performed with a reasonable coupling efficiency. Typically, coupling chemistry must be specifically developed for various PS that is unique for the chemistry of the PS and the carrier proteins that have been selected. This coupling chemistry introduces functional groups in the PS that then can be linked to carrier protein typically through the epsilon amino side chains of lysine residues. The chemical modification of PS to introduce such coupling groups can destroy epitopes on the PS and introduce new epitopes (e.g., associated with the linker or modified saccharide groups) whose significance can only be assessed by performing careful immunological analysis. Furthermore, for conventional PS-protein conjugate vaccines, the size of the PS, the number of PS molecules bound per protein carrier molecule, the nature of the carrier selected, and the type of linkage chemistry can all affect immunogenicity of the conjugate vaccine. As such, for example, in the case of pneumococcal disease where each of the 90+known serotypes has a different PS structure (Bentley et al., PLOS Genetics 2(3):e31 262-269, 2006), one single conjugation method may not be appropriate for all serotypes.

Reproducibly synthesizing conjugate vaccines with reproducible immunological properties involves careful control of the size of the PS, the number of PS molecules bound per protein carrier molecule, the nature of the carrier selected, and the type of linkage chemistry and this, in turn, dramatically increases the cost of manufacture of conjugate vaccines.

The emergence of antibiotic resistance highlights the urgency for the development of safe and effective vaccines. Making vaccines widely available, especially for those in developing countries, requires that the manufacture of vaccines also to be cost-effective. Incorporation of combined conjugate vaccines against many polysaccharide antigens from different serotypes of one or more bacterial species into the childhood immunization regimen would simplify vaccine administration in that high-risk population. However, current conjugate vaccine technology is not cost-effective and thus, combination conjugate vaccines are virtually impossible to deliver to the developing world because of the high cost.

In desirable embodiments, the immunogenic vaccine compositions of the invention are protein capsular matrix vaccines (PCMV) where one or more bacterial capsular components are entrapped in a crosslinked carrier protein matrix having a particle size range above 100 nm diameter, desirably in the range of 100 nm to 2000 nm diameter, or will include predominantly particles selected from within that range. PCMVs can be produced easily because one needs as a starting material the antigen of interest, e.g., capsules, that need not be hydrolyzed to smaller fragments and may enable multiple polysaccharides to become entrapped simultaneously.

Because the method of making vaccines of the invention does not require any knowledge of the chemistry of the antigen of interest, e.g., a capsular polysaccharide, the method does not depend on the need to develop cross-linking chemistry that is compatible with the chemistry of the antigen of interest and the carrier protein. While it is possible that some antigens may nonetheless interact with the crosslinker, this should not detract from the efficacy of the vaccine, because the unintended cross-linking of the antigen of interest and the carrier protein would be expected to have immunogenic properties anyway. In the vaccines of the invention, cross-linking of the antigen of interest to the carrier protein is not a requirement for the vaccine to be effective. This is in sharp contrast to conventional conjugate vaccines, which are thus hampered in their manufacture and development. The vaccines of the invention desirably have at least, e.g., 1%, 5%, 10%, 20%, 30%, 40%, 50%, 60%, 70%, 80%, 90%, 95%, 98%, or even 100% of the carrier proteins cross-linked and no more than, e.g., 50%, 40%, 30%, 25%, 20%, 15%, 10%, 5%, or 1% of the antigen of interest cross-linked to the carrier protein. Desirably, no more than 10% of antigens are cross-linked to the carrier proteins and at least 50% of carrier proteins are cross-linked.

As discussed herein, the protein matrix vaccine compositions of high molecular weight protein matrix particles according to the present invention have increased immunogenicity compared to compositions of low molecular weight protein matrix particles or compositions having a broad range of particle sizes including lower molecular weight protein matrix particles. Therefore, following admixing the antigen and carrier protein components and initiating a crosslinking reaction to cause crosslinking of the carrier protein, the reaction product is desirably further processed to eliminate lower molecular weight species (e.g., <100 nm diameter species) or by selecting the protein matrix particle sizes of the composition to include particle sizes greater than at least 100 nn diameter. Preferred compositions according to the invention will have a particle size range from 120-2000 nm diameter or will include predominantly particles selected from within that range. In desirable embodiments of the invention, the protein matrix vaccine compositions will have protein matrix particles of a mean particle size diameter greater than 120 nm, greater than 170 nm, greater than 200 nm, greater than 500 nm, greater than 1000 nm, greater than 2000 nm or even larger, e.g., to the limits of the methodology for collecting the protein matrix particles. In yet more desirable embodiments of the invention, the immunogenic compositions of the invention are comprised of protein matrix/antigen complexes having a range of particle sizes above 100 nm in diameter, such as 100-2000 nm diameter, or selections within that range, e.g., 120-200 nm, 200-400 nm, 250-500 nm, 120-1000 nm, 200-2000 nm, and other such particle size ranges. In yet further desirable embodiments of the invention, the composition includes complexes having particle sizes of 170-185 nm diameter. As discussed herein, raising the average complex particle size, or eliminating lower particle size components from the vaccine composition, leads to a surprising increase in immunogenicity with respect to the entrapped antigen. Moreover, larger protein matrix particles containing very small amounts of antigen are able to elicit immune responses surpassing or comparable to compositions of the antigen alone (uncomplexed) containing many times (e.g., 67-fold) more antigen than the particle size-selected protein capsular matrix composition of this invention.

Desired size particles can be fractionated by any suitable means, including size exclusion chromatography (SEC), followed by pooling the larger sized particles and discarding smaller sized particles. Alternatively, use of filter membranes with well chosen molecular weight cutoffs could be used to remove smaller sized particles while retaining particles of the desired size. The elimination of lower molecular weight species (e.g., <100 nm diameter species) or the selection the protein matrix particle sizes of the composition to include particle sizes greater than at least 100 nn diameter can be accomplished by any known means in the art, for example, chromatography, including size-exclusion chromatography (SEC), gel-filtration chromatography, or gel-permeation chromatography. Gel electrophoresis techniques could also be used.

The methods of making vaccines described herein do not result in the extensive modification of the antigen of interest, e.g., a capsular polymer. The antigen generally remains in the same state with a possible modification being, e.g., the reduction of reducing sugars for PS capsules that carry such groups at the end of the polymer chains. Such minor modifications are unlikely to affect immunogenicity of most capsular PS because the end sugars are 100-10000 times less abundant than the internal residues in the polymer. In contrast, for conventional conjugate vaccines, it is usually necessary to introduce linker groups into the antigen, e.g., a capsular polymer, that serve as the point of covalent attachment of the carrier protein. Linkers need to be used because many antigens, e.g., capsular polymers, do not have a reactive group such as a carboxyl or amino group as part of their structure. For example, the introduction reactive groups into a PS can result in destruction of capsular epitopes and generation of novel epitopes that might be undesirable in a vaccine product because of their unknown immunological cross-reactivity with host self-epitopes.

The methods of making vaccines described herein are less complex than conjugate vaccine technology because its chemistry depends only on the cross-linking chemistry of the carrier protein (e.g., DNI, cholera toxin B subunit, diphtheria toxoid, tetanus toxoid or Fragment C, or Escherichia coli beta-galactosidase). For example, while the capsular polymer affects the rate of cross-linking when mixed with DNI, it does not affect the pattern or extent of cross-linking which is governed more by the protein being used, its concentration, and the concentration of the cross-linking agent (e.g., glutaraldehyde) added. These parameters can readily be adjusted, thereby reducing the time and effort required to make the vaccine, and saving expense.

The methods of making PCMV compositions described herein can be used with any antigen, e.g., a capsular polymer or any biopolymer with few if any amino groups, and any carrier protein that can be crosslinked, e.g., carrier proteins not having critical epitopes that can be destroyed by borohydride reduction. Carrier proteins that may be used in the methods described herein desirably have at least 2 lysine residues or other residues that are unblocked and that can be crosslinked by chemical modification. Tetanus toxoid is one possible carrier protein. This toxin is rendered non-toxic by treatment with formaldehyde, a reagent that reacts with amino groups of proteins. Other desirable carrier proteins include the cholera toxin B subunit (available from SBL Vaccin AB), diphtheria toxoid or CRM197, tetanus toxoid or Fragment C (available from Sigma Aldrich), DNI, or beta-galactosidase from Escherichia coli (available from Sigma Aldrich).

Current multivalent conjugate vaccines are made by synthesis of individual conjugate vaccines first, followed by their mixing to produce a “cocktail” conjugate vaccine (e.g., the Wyeth hepta-valent pneumococcal vaccine, Prevnar®). The present invention's methods of making vaccines can be used to make multivalent vaccines by mixing chemically different antigens, e.g., capsular organic polymers, together before crosslinking the carrier protein, e.g., with glutaraldehyde or other crosslinking agent, or by mixing specific vaccines of the invention that were synthesized separately. This flexibility provides significant advantages over conventional methods of manufacturing multivalent vaccines.

Exemplary vaccines of the invention discussed in the examples performed comparably to conjugate vaccines despite the fact that these vaccines were synthesized by a method that is not predicted to generate any covalent bonds between atoms making up the antigen molecule and the carrier protein. Glutaraldehyde reacts exclusively with amino side chains of proteins typified by the epsilon amino group of lysine residues. Polysaccharide antigens contain few free amino groups (any amino side chains are typically acetylated) to react with glutaraldehyde or aldehyde-functional crosslinkers (e.g., OCH—R—CHO, discussed supra), therefore such antigens are well suited to PCMV formation, where less than 50% of antigen is crosslinked directly to a carrier protein. As seen in the examples below, the immune responses generated by PCMVs, which compared favorably to conjugate controls, indicate that PS molecules were molecularly entrapped within a crosslinked matrix of DNI protein molecules.

According to a non-limiting model, the entrapment acts to carry the protein matrix vaccine composition into B cells that bind such matrixes by virtue of Ig receptors that recognize PGA immunologically. Once taken up inside these B cells, the matrixes are degraded in a manner similar to conventional conjugate vaccines and that this results in carrier protein-derived peptides that are displayed on MHC class II molecules of the corresponding B cells. This in turn recruits T cell help and thus leads to the expansion and maturation of such B cells to become IgG producing plasma and memory cells specific for the antigen. Thus, according to the non-limiting model PCMVs work like protein-conjugate capsular vaccines immunologically but are distinct because PCMVs lack significant covalent bonding between the carrier protein and the capsular polymers.

The vaccines of the invention, including PCMVs, may be used in combination, for example, in pediatric vaccines. In addition, the vaccines of the invention may be used to vaccinate against, for example, pneumococcal infection, streptococcal (groups A and B) infection, Haemophilus influenzae type B (“HiB”) infection, meningococcal (e.g., Neisseria meningitides) infection, and may be used as O antigen vaccines from Gram negative bacteria (e.g., Pseudomonas aeruginosa, Francisella tularensis (Thirumalapura et al., J. Med. Microbiol. 54:693-695, 2005; Vinogradov and Perry, Carbohydr. Res. 339:1643-1648, 2004; Vinogradov et al., Carbohydr. Res. 214:289-297, 1991), Shigella species, Salmonella species, Acinetobacter species, Burkholderia species, and Escherichia coli.

Vaccines of the invention may be made using any linkers, such as, e.g., those described herein, to crosslink any carrier protein, such as, e.g., those described herein, in the presence of one or more antigens of interest, such as, e.g., those described herein. If one antigen of interest is used, the protein matrix vaccine of the invention is said to be monovalent. If more than one antigen of interest is used, the protein matrix vaccine of the invention is said to be multivalent. If a microbial capsular polymer or polysaccharide is the antigen of interest, the protein matrix vaccine of the invention is said to be a protein capsular matrix vaccine (PCMV).

### Linkers

Crosslinking agents useful to crosslink carrier proteins are well known in the art and include glutaraldehyde, m-maleimidobenzoyl-N-hydroxysuccinimide ester, carbodiimide, and bis-biazotized benzidine.

General methods and moieties for directly crosslinking carrier proteins, using a homobifunctional or a heterobifunctional linker are described, for example, by G. T. Hermanson, Bioconjugate Techniques (Academic Press, 1996) and Dick and Beurret, “Glycoconjugates of Bacterial Carbohydrate Antigens,” in Conjugate Vaccines (Cruse and Lewis, eds), Contrib. Microbiol. Immunol. Basel, Karger, 1989, vol. 10, pp. 48-114). For example, with a carrier protein possessing n number of lysine moieties, there are, theoretically, n+1 primary amines (including the terminal amine) available for reaction with an exemplary crosslinker's carboxylic group. Thus, using this direct conjugation procedure the product is limited to having n+1 amide bonds formed.

The linker employed in desirable embodiments of the present invention is, at its simplest, a bond connecting two carrier proteins. The linker can be, a linear, cyclic, or branched molecular skeleton, with pendant groups which bind covalently to two carrier proteins, (A) and (B). Any given carrier protein may be linked to more than one carrier protein, such that a matrix of interconnected carrier proteins is created, in which an antigen of interest may be entrapped.

The term “linkage group” refers to the covalent bond that results from the combination of reactive moieties of linker (L) with functional groups of (A) or (B). Examples of linkage groups include, without limitation, ester, carbamate, thioester, imine, disulfide, amide, ether, thioether, sulfonamide, isourea, isothiourea, imidoester, amidine, phosphoramidate, phosphodiester, thioether, and hydrazone.

The linking of (A) with (B) is achieved by covalent means, involving bond (linkage group) formation with one or more functional groups located on (A) and (B). Examples of chemically reactive functional groups which may be employed for this purpose include, without limitation, amino, hydroxyl, sulfhydryl, carboxyl, carbonyl, thioethers, guanidinyl, imidazolyl, and phenolic groups, all of which are present in naturally-occurring amino acids in many carrier proteins.

The covalent linking of (A) with (B) may therefore be effected using a linker (L) which contains reactive moieties capable of reaction with such functional groups present in (A) and (B). The product of this reaction is a linkage group which contains the newly formed bonds linking (L) with (A) and (L) with (B). For example, a hydroxyl group of (A) may react with a carboxylic acid group of (L), or an activated derivative thereof, vide infra, resulting in the formation of an ester linkage group.

Examples of moieties capable of reaction with sulfhydryl groups include α-haloacetyl compounds of the type XCH₂CO— (where X=Br, Cl, or I), which show particular reactivity for sulfhydryl groups, but which can also be used to modify imidazolyl, thioether, phenol, and amino groups as described by, for example, Gurd, Methods Enzymol., 11:532, 1967. N-Maleimide derivatives are also considered selective towards sulfhydryl groups, but may additionally be useful in coupling to amino groups under certain conditions. Reagents such as 2-iminothiolane (Traut et al., Biochemistry, 12:3266, 1973), which introduce a thiol group through conversion of an amino group, may be considered as sulfhydryl reagents if linking occurs through the formation of disulphide bridges.

Examples of reactive moieties capable of reaction with amino groups include, for example, alkylating and acylating agents. Representative alkylating agents include:

(i) α-haloacetyl compounds, which show specificity towards amino groups in the absence of reactive thiol groups and are of the type XCH₂CO— (where X=Cl, Br or D as described by, for example, Wong (Biochemistry, 24:5337, 1979);
(ii) N-maleimide derivatives, which may react with amino groups either through a Michael type reaction or through acylation by addition to the ring carbonyl group as described by, for example, Smyth et al. (J. Am. Chem. Soc., 82:4600, 1960 and Biochem. J., 91:589, 1964);
(iii) aryl halides such as reactive nitrohaloaromatic compounds;
(iv) alkyl halides, as described by, for example, McKenzie et al. (J. Protein Chem., 7:581, 1988);
(v) aldehydes and ketones capable of Schiffs base formation with amino groups, the adducts formed usually being stabilized through reduction to give a stable amide;
(vi) epoxide derivatives such as epichlorohydrin and bisoxiranes, which may react with amino, sulfhydryl, or phenolic hydroxyl groups;
(vii) chlorine-containing derivatives of s-triazines, which are very reactive towards nucleophiles such as amino, sulfhydryl, and hydroxyl groups;
(viii) aziridines based on s-triazine compounds detailed above as described by, for example, Ross (J. Adv. Cancer Res., 2:1, 1954), which react with nucleophiles such as amino groups by ring opening;
(ix) squaric acid diethyl esters as described by, for example, Tietze (Chem. Ber., 124:1215, 1991); and
(x) α-haloalkyl ethers, which are more reactive alkylating agents than normal alkyl halides because of the activation caused by the ether oxygen atom, as described by, for example, Benneche et al. (Eur. J. Med. Chem., 28:463, 1993).

Representative amino-reactive acylating agents include:

(i) isocyanates and isothiocyanates, particularly aromatic derivatives, which form stable urea and thiourea derivatives respectively;
(ii) sulfonyl chlorides, which have been described by, for example, Herzig et al. (Biopolymers, 2:349, 1964);
(iii) acid halides;
(iv) active esters such as nitrophenylesters or N-hydroxysuccinimidyl esters;
(v) acid anhydrides such as mixed, symmetrical, or N-carboxyanhydrides;
(vi) other useful reagents for amide bond formation as described by, for example, M. Bodansky (Principles of Peptide Synthesis, Springer-Verlag, 1984);
(vii) acylazides, e.g., where the azide group is generated from a preformed hydrazide derivative using sodium nitrite, as described by, for example, Wetz et al. (Anal. Biochem., 58:347, 1974); and
(viii) imidoesters, which form stable amidines on reaction with amino groups as described by, for example, Hunter and Ludwig (J. Am. Chem. Soc., 84:3491, 1962).

Aldehydes, such as, e.g., glutaraldehyde, and ketones may be reacted with amines to form Schiff's bases, which may advantageously be stabilized through reductive amination. Alkoxylamino moieties readily react with ketones and aldehydes to produce stable alkoxyamines as described by, for example, Webb et al. (Bioconjugate Chem., 1:96, 1990).

Examples of reactive moieties capable of reaction with carboxyl groups include diazo compounds such as diazoacetate esters and diazoacetamides, which react with high specificity to generate ester groups as described by, for example, Herriot (Adv. Protein Chem., 3:169, 1947). Carboxylic acid modifying reagents such as carbodiimides, which react through O-acylurea formation followed by amide bond formation, may also be employed.

The functional groups in (A) and/or (B) may, if desired, be converted to other functional groups prior to reaction, for example, to confer additional reactivity or selectivity. Examples of methods useful for this purpose include conversion of amines to carboxylic acids using reagents such as dicarboxylic anhydrides; conversion of amines to thiols using reagents such as N-acetylhomocysteine thiolactone, S-acetylmercaptosuccinic anhydride, 2-iminothiolane, or thiol-containing succinimidyl derivatives; conversion of thiols to carboxylic acids using reagents such as .alpha.-haloacetates; conversion of thiols to amines using reagents such as ethylenimine or 2-bromoethylamine; conversion of carboxylic acids to amines using reagents such as carbodiimides followed by diamines; and conversion of alcohols to thiols using reagents such as tosyl chloride followed by transesterification with thioacetate and hydrolysis to the thiol with sodium acetate.

So-called zero-length linkers, involving direct covalent joining of a reactive chemical group of (A) with a reactive chemical group of (B) without introducing additional linking material may, if desired, be used in accordance with the invention. Examples include compounds in which (L) represents a chemical bond linking an oxygen atom of (A) to a carbonyl or thiocarbonyl moiety present in (B), such that the linkage group is an ester or thioester. For example, an amino group (A) can be linked to a carboxyl group (B) by using carbodiimide chemistry yielding A-L-B where L is a amide bond or RC(:O) linked to N—R where R is the carbon chain derived from amino acid side chains of the same or two different protein molecules. Most commonly, however, the linker includes two or more reactive moieties, as described above, connected by a spacer element. The presence of a spacer permits bifunctional linkers to react with specific functional groups within (A) and (B), resulting in a covalent linkage between these two compounds. The reactive moieties in a linker (L) may be the same (homobifunctional linker) or different (heterobifunctional linker, or, where several dissimilar reactive moieties are present, heteromultifunctional linker), providing a diversity of potential reagents that may bring about covalent attachment between (A) and (B).

Spacer elements typically consist of chains which effectively separate (A) and (B) by a linear or branched alkyl of 1 to 10 carbon atoms, a linear or branched heteroalkyl of 1 to 10 atoms, a linear or branched alkene of 2 to 10 carbon atoms, a linear or branched alkyne of 2 to 10 carbon atoms, an aromatic residue of 5 to 10 carbon atoms, a cyclic system of 3 to 10 atoms, or —(CH₂CH₂O)ₙCH₂CH₂—, in which n is 1 to 4.

The nature of extrinsic material introduced by the linking agent may have a bearing on the pharmacokinetics and/or activity of the ultimate vaccine product. Thus it may be desirable to introduce cleavable linkers, containing spacer arms which are biodegradable or chemically sensitive or which incorporate enzymatic cleavage sites.

These cleavable linkers, as described, for example, in PCT Publication WO 92/17436 (hereby incorporated by reference), are readily biodegraded in vivo. In some cases, linkage groups are cleaved in the presence of esterases, but are stable in the absence of such enzymes. (A) and (B) may, therefore, advantageously be linked to permit their slow release by enzymes active near the site of disease.

Linkers may form linkage groups with biodegradable diester, diamide, or dicarbamate groups of the formula: —(Z¹)ₒ—(Y¹)ᵤ—(Z²)ₛ—(R₁₁)—(Z³)ₜ—(Y²)ᵥ—(Z⁴)ₚ— wherein each of Z¹, Z², Z³, and Z⁴ is independently selected from O, S, and NR₁₂ (where R₁₂ is hydrogen or an alkyl group); each of Y¹ and Y² is independently selected from a carbonyl, thiocarbonyl, sulphonyl, phosphoryl or similar acid-forming group; o, p, s, t, u, and v are each independently 0 or 1; and R₁₁ is a linear or branched alkyl of 1 to 10 carbon atoms, a linear or branched heteroalkyl of 1 to 10 atoms, a linear or branched alkene of 2 to 10 carbon atoms, a linear or branched alkyne of 2 to 10 carbon atoms, an aromatic residue of 5 to 10 carbon atoms, a cyclic system of 3 to 10 atoms, —(CH₂CH₂O)_{q}CH₂CH₂— in which q is 1 to 4, or a chemical bond linking —(Z¹)ₒ—(Y¹)ᵤ—(Z²)ₛ—(R₁₁)—(Z³)ₜ—(Y²)ᵥ—(Z⁴)ₚ—.

Exemplary desirable linkers (L) used in the present invention may be described by any of formulas I-II:

—C:O—R₁₃—C:O— I

—C:O—NH—R₁₃—NH—C:O— II

where the linker is covalently attached to both an oxygen atom (A) and an oxygen atom of (B). Accordingly, linker (L) of formulas I-II are attached to carrier proteins (A) and (B) via dipyran, ester, or carbamate linkage groups. In these embodiments, R₁₃ represents a linear or branched alkyl of 1 to 10 carbon atoms, a linear or branched heteroalkyl of 1 to 10 atoms, a linear or branched alkene of 2 to 10 carbon atoms, a linear or branched alkyne of 2 to 10 carbon atoms, an aromatic residue of 5 to 10 carbon atoms, a cyclic system of 3 to 10 atoms, —(CH₂CH₂O)ₙCH₂CH₂— in which n is 1 to 4, or a chemical bond linking two nitrogens or two carbonyls.

Linkers designed to form hydrazone linkages have the chemical formula III:

—(Y³)—(Z⁵)_{w}—R₁₄—C(:X₄)—R₁₅ III

where Z⁵ is selected from O, S, or NR₁₆; R₁₆ is hydrogen or an alkyl group; R₁₅ is selected from hydrogen, an alkyl, or a heteroalkyl; Y³ is selected from a carbonyl, thiocarbonyl, sulphonyl, phosphoryl, or a similar acid-forming group covalently bound to an oxygen atom of (A); w is 0 or 1; R₁₄ is a linear or branched alkyl of 1 to 10 carbon atoms, a linear or branched heteroalkyl of 1 to 10 atoms, a linear or branched alkene of 2 to 10 carbon atoms, a linear or branched alkyne of 2 to 10 carbon atoms, an aromatic residue of 5 to 10 carbon atoms, a cyclic system of 3 to 10 atoms, —(CH₂CH₂O)ₙCH₂CH₂—, in which n is 1 to 4, or a chemical bond linking —(Y³)—(Z⁵)_{w}— to and X₄ is a hydrazone resulting from the condensation reaction of (B) containing a hydrazide group and the precursor to linker II, in which X₄ is the oxygen atom of a ketone or aldehyde group.

### Carrier Proteins

In general, any carrier protein that can entrap an antigen under physiological conditions may be used in the present invention. Desirably, the antigen is entrapped in a complex with crosslinked carrier protein in the absence of significant covalent bonding between the antigen and the carrier protein. Absence of significant covalent bonding, refers to no more than 50% of the antigen being covalently bonded to a carrier protein. In desirable embodiments, no more than 40%, 30%, 10%, or 5% of the antigen is covalently bonded to a carrier protein. The antigen/carrier protein complex may contain another compound, such as alum, and this other compound, in desirable embodiments, can entrap the antigen and carrier protein.

Carrier proteins used in the vaccines of the invention desirably are proteins that, either alone or in combination with an antigen, elicit an immune response in a subject. Desirably, the carrier protein contains multiple MCH class II-restricted epitopes recognized by a helper T cell. Desirably, the epitopes are capable of inducing a Tₕ cell response in a subject and induce B cells to produce antibodies against the entire antigen of interest. Epitopes as used in describing this invention, include any determinant on an antigen that is responsible for its specific interaction with an antibody molecule or fragment thereof. Epitopic determinants usually consist of chemically active surface groupings of molecules such as amino acids or sugar side chains and have specific three-dimensional structural characteristics as well as specific charge characteristics. To have immunogenic properties, a protein or polypeptide generally is capable of stimulating T cells. However, a carrier protein that lacks an epitope recognized by a T cell may also be immunogenic.

By selecting a carrier protein which is known to elicit a strong immune response (i.e., is highly immunogenic), a diverse population of subjects can be treated by a protein matrix vaccine composition described herein. The carrier protein desirably is sufficiently foreign to elicit a strong immune response to the vaccine. Typically, the carrier protein used is a molecule that is capable of imparting immunogenicity to the antigen of interest. In a desirable embodiment, a carrier protein is one that is inherently highly immunogenic. Thus a carrier protein that has a high degree of immunogenicity and is able to maximize antibody production to the antigen(s) complexed with it is desirable.

Various carrier proteins of the invention include, e.g., toxins and toxoids (chemical or genetic), which may or may not be mutant, such as anthrax toxin, PA and DNI (PharmAthene, Inc.), diphtheria toxoid (Massachusetts State Biological Labs; Serum Institute of India, Ltd.) or CRM197, tetanus toxin, tetanus toxoid (Massachusetts State Biological Labs; Serum Institute of India, Ltd.), tetanus toxin fragment Z, exotoxin A or mutants of exotoxin A of Pseudomonas aeruginosa, bacterial flagellin, pneumolysin, an outer membrane protein of Neisseria meningitidis (strain available from the ATCC (American Type Culture Collection, Manassas, Va.)), Pseudomonas aeruginosa Hcp1 protein, Escherichia coli heat labile enterotoxin, shiga-like toxin, human LTB protein, a protein extract from whole bacterial cells, and any other protein that can be cross-linked by a linker. Desirably, the carrier protein is the cholera toxin B subunit (available from SBL Vaccin AB), diphtheria toxoid or CRM197 (Connaught, Inc.), tetanus toxoid or Fragment C (available from Sigma Aldrich), DNI, or beta-galactosidase from E. coli (available from Sigma Aldrich). Other desirable carrier proteins include bovine serum albumin (BSA), P40, and chicken riboflavin. (Unless otherwise indicated, the exemplary carrier proteins are commercially available from Sigma Aldrich.) Other exemplary carrier proteins are MAPs (multi-antigenic peptides), which are branched peptides. By using a MAP, crosslinking density is maximized because of multiple branched amino acid residues. A desirable amino acid residue for crosslinking purposes, which can be used to form a MAP, is, but is not limited to, lysine, having a free amino group on its side chain.

Both BSA and keyhole limpet hemocyanin (KLH) have commonly been used as carriers in the development of vaccines when experimenting with animals. Carrier proteins which have been used in the preparation of therapeutic vaccines include, but are not limited to, a number of toxins of pathogenic bacteria and their toxoids. Examples include diphtheria and tetanus toxins and their medically acceptable corresponding toxoids. Other candidates are proteins antigenically similar to bacterial toxins referred to as cross-reacting materials (CRMs). Carrier proteins useful in the practice of the invention may also include any protein not derived from humans and not present in any human food substance.

In desirable embodiments of the invention, proteins that form ring-like structures are used for PCMV production. Such proteins include the Hcp1 protein of Pseudomonas aeruginosa, the nontoxic “B subunits” of cholera toxin, the heat-labile enterotoxin of Escherichia coli, and shiga-like toxin. Such ring-like protein complexes can form “beads on a string” where the linear PS chains penetrate the central channel of these ring-shaped protein complexes. After protein cross-linking, such complexes are predicted to be particularly stable. Structural data of the proteins suggest these central channels are large enough for PS chains to enter easily. For example, the central channel of the Hcp1 hexameric ring is 42 Angstroms which is wide enough to easily accommodate several polysaccharide chains of 5.5 Angstroms in width (Mougous et al., Science, 312(5779):1526-1530 (2006)). Alternatively, protein rings may be assembled around the PS (e.g., from subunits of a monomeric carrier protein that naturally assemble into rings under particular physical chemical conditions). Such monomeric proteins that can assemble into rings are known in the art and include, for example, pneumolysin (Walker et al., Infect. Immun., 55(5):1184-1189 (1987); Kanclerski and Mollby, J. Clin. Microbiol., 25(2):222-225 (1987)), listeriolysin O (Kayal and Charbit, FEMS Microbiol. Rev., 30:514-529 (2006); Mengaud et al., Infect. Immun., 55(12):3225-3227 (1987)), DNI, anthrax PA, Hcp1, cholera toxin B subunit, shiga toxin B subunit, flagellin, and numerous related molecules known in the art and made by various microorganisms.

In another desirable embodiment, Toll-like receptor (TLR) agonists are used as carrier proteins. Toll-like receptor (TLR) activation is important in shaping the adaptive immune response and may play a role in affinity maturation of the antibody response, isotype switching, and immunological memory. Flagellin (FLA) of Vibrio cholerae is a TLR agonist. Over 20 mgs of FLA protein has been purified from recombinant Escherichia coli and shown to be a potent TLR activator in an IL-6 macrophage induction assay. In addition, a well-conserved Streptococcus pneumoniae protein called “Pneumolysin” has also been shown to activate TLR4 and, additionally, is a protective antigen. Thus, this protein can also be used as a protein matrix carrier protein.

Further, outer membrane protein (OMP) mixtures (e.g., the OMPs of Neisseria meningitidis) are used as the carrier protein for HIB conjugate vaccine produce by Merck and protein extracts from whole Streptococcal pneumoniae bacterial cells have been shown to be at least partially protective in animal infection models. In desirable embodiments of the invention, these protein mixtures may be used as carrier proteins.

In a desirable embodiment, the vaccine composition is made using a carrier protein that has, e.g., at least two lysine residues or other residues that are unblocked and that can be cross-linked by chemical modification. In other desirable embodiments, the carrier protein is a multimer (e.g., one containing at least 5 subunits).

In another embodiment, DNI is used as the carrier protein because it is nontoxic, leaving no need to render it less toxic before use. Furthermore, the use of DNI is desirable because DNI may also induce a protective immune response to B. anthracis, in addition to the protective immune response elicited to the antigen of interest. Also, DNI has no internal disulfide bonds. Such bonds are susceptible to borohydride reduction, which could denature the protein and result in loss of epitopes that induce anthrax toxin neutralizing antibody.

### Antigens of Interest

The vaccine compositions of the invention and methods of making and administering such vaccines can be used for any antigen of interest, e.g., a polysaccharide, polyalcohol, or poly amino acid. Desirably, the antigen of interest carries no primary groups that can be destroyed by the chemical reactions employed by the method of making vaccines, e.g., the denaturing of an antigen caused by the destruction of antigen disulfide bonds by borohydride reduction. Exemplary antigens of interest include but are not limited to organic polymers such as polysaccharides (e.g., polysaccharides having at least 18 residues), phosphopolysaccharides, polysaccharides with amino sugars with N-acetyl substitutions, polysaccharides containing sulfonylated sugars, other sulfate-modified sugars, or phosphate-modified sugars, polyalcohols, poly amino acids, teichoic acids, O side chains of lipopolysaccharides. Exemplary antigens of interest also include capsular organic polymers including those synthesized by microbes, e.g., bacteria, fungi, parasites, and viruses, and then purified from such a biological source using standard methods. Exemplary antigens of interest include microbial capsular organic polymers including those purified from bacterial organisms such as Bacillus species (including B. anthracis) (Wang and Lucas, Infect. Immun., 72(9):5460-5463 (2004)), Streptococcus pneumoniae (Bentley et al., PLoS Genet., 2(3):e31 (2006); Kolkman et al., J. Biochemistry, 123:937-945 (1998); and Kong et al., J. Med. Microbiol., 54:351-356 (2005)), Shigella (Zhao et al., Carbohydr. Res., 342(9):1275-1279 (2007)), Haemophilus influenzae, Neisseria meningitidis, Staphylococcus aureus, Salmonella typhi, Streptococcus pyogenes, Escherichia coli (Zhao et al., Carbohydr. Res., 342(9):1275-1279 (2007)), and Pseudomonas aeruginosa, and fungal organisms such as Cryptococcus and Candida, as well as many other microorganisms (see, e.g., Ovodov, Biochemistry (Mosc.), 71(9):937-954 (2006); Lee et al., Adv. Exp. Med. Biol., 491:453-471 (2001); and Lee, Mol. Immunol., 24(10):1005-1019 (1987)). Exemplary antigens of interest also include polymers that do not occur in nature and thus are non-biological in origin.

Particular Streptococcus pneumoniae antigens include polysaccharide capsular type 1 (e.g., 1-g or 1-q), 2 (e.g., 2-g, 2-q, or 2-41A), 3 (e.g., 3-g, 3-q, 3-c, or 3-nz), 4, 5 (e.g., 5-q, 5-c, 5-qap, or 5-g), 6A (e.g., 6A-g, 6A-cl, 6A-c2, 6A-n, 6A-qap, 6A-6B-g, 6A-6B-q, or 6A-6B-s), 6B (e.g., 6B-c, 6A-6B-g, 6A-6B-q, or 6A-6B-s), 7F (e.g., 7F-7A), 7A (e.g., 7A-cn or 7F-7A), 7B (e.g., 7B-40), 7C (e.g., 7C-19C-24B), 8 (e.g., 8-g or 8-s), 9A (e.g., 9A-9V), 9L, 9N, 9V (e.g., 9A-9V), 9V and 14, 1° F. (e.g., 10E-q, 10E-ca, or 10E-10C), 10A (e.g., 10A-17A or 10A-23F), 10B (e.g., 10B-10C), 11F, 11A (e.g., 11A-nz or 11A-11D-18F), 11B (e.g., 11B-11C), 11C (e.g., 11B-11C. or 11C-cn), 11D (e.g., 11A-11D-18F), 12F (e.g., 12F-q or 12F-12A-12B), 12A (e.g., 12A-cn, 12A-46, or 12F-12A-12B), 12B (e.g., 12F-12A-12B), 13 (e.g., 13-20), 14 (e.g., 14-g, 14-q, 14-v, or 14-c), 15F (e.g., 15F-cn1 or 15F-cn2), 15A (e.g., 15A-ca1, 15A-ca2, or 15A-chw), 15B (e.g., 15B-c, 15B-15C, 15B-15C-22F-22A), 15C (e.g., 15C-ca, 15C-q1, 15C-q2, 15C-q3, 15C-s, 15B-15C, or 15B-15C-22F-22A), 16F (e.g., 16F-q or 16F-nz), 16A, 17F (e.g., 17F-n and 17F-35B-35C-42), 17A (e.g., 17A-ca or 10A-17A), 18F (e.g., 18F-ca, 18F-w, or 11A-11D-18F), 18A (e.g., 18A-nz or 18A-q), 18B (e.g., 18B-18C), 18C (e.g., 18B-18C), 19F (e.g., 19F-g1, 19F-g2, 19F-g3, 19F-q, 19F-n, or 19F-c), 19A (e.g., 19A-g, 19A-, or 19A-ca), 19B, 19C (e.g., 19C-cn1, 19C-cn2, or 7C-19C-24B), (e.g., 13-20), 21 (e.g., 21-ca or 21-cn), 22F (e.g., 15B-15C-22F-22A), 23F (e.g., 23F-c, 10A-23F, or 23F-23A), 23B (e.g., 23B-c or 23B-q), 24F (e.g., 24F-cn1, 24F-cn2, or 24F-cn3), 24A, 24B (e.g., 7C-19C-24B), 25F (e.g., 25F-38), 25A, 27, 28F (e.g., 28F-28A or 28F-cn), 28A (e.g., 28F-28A), 29 (e.g., 29-ca or 29-q), 31, 32F (e.g., 32F-32A), 32A (e.g., 32A-cn or 32F-32A), 33F (e.g., 33F-g, 33F-q, 33F-chw, 33F-33B, or 33F-33A-35A), 33A (e.g., 33F-33A-35A), 33B (e.g., 33B-q, 33B-s, or 33F-33B), 33D, 34 (e.g., 34-ca or 34s), 35F (e.g., 35F-47F), 35A (e.g., 33F-33A-35A), 35B (e.g., 17F-35B-35C-42), 36, 37 (e.g., 37-g or 37-ca), 38 (e.g., 25F-38), 39 (e.g., 39-cn1 or 39-cn2), 40 (e.g., 7B-40), 41F (e.g., 41F-cn or 41F-s), 41A (e.g., 2-41A), 42 (e.g., 17B-35B-35C-42), 43, 44, 45, 46 (e.g., 46-s or 12A-46), 47F (e.g., 35F-47F), 47A, 48 (e.g., 48-cn1 or 48-cn2), or GenBank Accession Number AF532714 or AF532715.

Particular mention is made of Streptococcus pneumoniae polysaccharides selected from the group consisting of capsular type 3, 4, 6B, 7A, 7B, 7C, 7F, 9A, 9L, 9N, 9V, 12A, 12B, 12F, 14, 15A, 15B, 15C, 15F, 17, 18B, 18C, 19F, 23F, 25A, 25F, 33F, 35, 37, 38, 44, or 46.

### Vaccine Compositions

The vaccine compositions of the invention, including PCMVs, may be used in combination, for example, in pediatric vaccines. In addition, the vaccine compositions of the invention may be used to vaccinate against, for example, Pneumococcus infection, Haemophilus influenzae type B (“HiB”) infection, Streptococcus (groups A and B) infection, meningococcal (e.g., Neisseria meningitides) infection, and may be used as O antigen vaccines from Gram negative bacteria (e.g., Pseudomonas aeruginosa, Francisella tularensis, Shigella species, Salmonella species, Acinetobacter species, Burkholderia species, and Escherichia coli).

The vaccine formulation desirably includes at least one carrier protein, one or more antigen of interest, and a pharmaceutically acceptable carrier or excipient (e.g., aluminum phosphate, sodium chloride, sterile water). A vaccine composition may also include an adjuvant system for enhancing the immunogenicity of the formulation, such as oil in a water system and other systems known in the art or other pharmaceutically acceptable excipients. A carrier/antigen complex that is insoluble under physiological conditions is desirable to slowly release the antigen after administration to a subject. Such a complex desirably is delivered in a suspension containing pharmaceutically acceptable excipients. However, the carrier/antigen complex may also be soluble under physiological conditions.

Typically the protein matrix vaccine is in a volume of about 0.5 ml for subcutaneous injection, 0.5 ml for intramuscular injection, 0.1 ml for intradermal injection, or 0.002-0.02 ml for percutaneous administration. A 0.5 ml dose of the protein matrix vaccine may contain approximately 2-500 μg of the antigen entrapped with approximately 2-500 μg of the carrier protein. In a desirable embodiment, in a 0.5 ml dose, approximately 10 μg of the antigen are entrapped with approximately 10 μg of the carrier protein. The molar ratio of antigen to carrier protein desirably is between 1 to 10 (e.g., 1 part antigen to 2 parts carrier or 1 part antigen to 3 parts carrier, etc.) and 10 to 1 (e.g., 3 parts antigen to 1 part carrier or 2 parts antigen to 1 part carrier, etc.). In a desirable embodiment, the molar ratio of antigen to carrier is 1 to 1. Alternatively, the ratio by dry weight of antigen to carrier protein desirably is between 1 to 10 and 10 to 1 (e.g., 1 to 1 by dry weight).

Because the peptides or conjugates may be degraded in the stomach, the vaccine is desirably administered parenterally (for instance, by subcutaneous, intramuscular, intravenous, intraperitoneal, or intradermal injection). While delivery by a means that physically penetrates the dermal layer is desirable (e.g., a needle, airgun, or abrasion), the vaccines of the invention can also be administered by transdermal absorption.

In particular, the vaccines of the invention may be administered to a subject, e.g., by intramuscular injection, intradermal injection, or transcutaneous immunization with appropriate immune adjuvants. Vaccines of the invention may be administered, one or more times, often including a second administration designed to boost production of antibodies in a subject to prevent infection by an infectious agent corresponding to the antigen included in the vaccine. The frequency and quantity of vaccine dosage to obtain the desired immune response or level of immunity depends on the specific activity of the vaccine and can be readily determined by routine experimentation. For example, for an infant, a vaccine schedule may be three doses of 0.5 ml each at approximately four to eight week intervals (starting at two months of age) followed by a fourth dose of 0.5 ml at approximately twelve to fifteen months of age. A fifth dose between four and six years of age may be desirable for some vaccines.

While the age at which the first dosage is administered generally is two months, a vaccine may be administered to infants as young as 6 weeks of age. For adults, two or more 0.5 ml doses given at internals of 2-8 weeks in between generally are sufficient to provide long-term protection. A booster dose is desirably given every ten years to previously immunized adults and children above eleven years of age.

The formulations may be presented in unit-dose or multi-dose containers, for example, sealed ampoules and vials and may be stored in a freeze-dried (lyophilized) condition requiring only the addition of the sterile liquid carrier immediately prior to use. Vaccines of the invention can be formulated in pharmacologically acceptable vehicles, e.g., alum hydroxide gel, adjuvant preparation, or saline, and then administered, e.g., by intramuscular injection, intradermal injection, or transcutaneous immunization with appropriate immune adjuvants.

The invention also includes kits that include a vaccine described herein (e.g., a PCMV). The kits of the invention can also include instructions for using the kits in the vaccination methods described herein.

The efficacy of the immunization schedule may be determined by using standard methods for measuring the antibody titer in the subject. In general, mean antibody titers (desirably IgG titers) of approximately 1 μg/ml are considered indicative of long-term protection.

The invention is described herein below by reference to specific examples, embodiments and figures, the purpose of which is to illustrate the invention rather than to limit its scope. The following examples are not to be construed as limiting.

The invention provides vaccine compositions containing an antigen of interest entrapped with a carrier protein matrix, methods of making such vaccines, and methods of vaccine administration. It has been discovered that the immunogenicity of the composition, and hence their effectiveness as vaccines, may be improved by controlling or selecting the particle size of the carrier protein matrix.

### Example 1

The effect of particle sizing on a matrix vaccine composition was investigated using as an antigen S. pneumoniae polysaccharide type 14 capsular polysaccharide (PPS-14) and using as a carrier protein the dominant negative mutant (DNI) form of B. anthracis protective antigen (PA) expressed from Escherichia coli as described by Benson et al. (Biochemistry, 37:3941-3948 (1998)).

The polysaccharide antigen (PPS 14) and carrier protein (DNI) were mixed at a 1:1 weight ratio and were present at 7.5 mg/ml for each component. Crosslinking of the DNI carrier protein was initiated by adding glutaraldehyde as a crosslinking agent. Two crosslinking reaction mixtures were made up: one having a final glutaraldehyde concentration of 0.05% and one having a final glutaraldehyde concentration of 0.25%. The crosslinking reaction was carried out in a total volume of 0.5 ml by incubating at 4° C. for 23 hours. At that time, sodium cyanoborohyride, which reduces Schiff bases, was added to a concentration of 20 mg/ml and the reaction mixture was incubated an additional hour.

A portion of the 0.25% glutaraldehyde reaction mixture was applied to a 25 ml Sepharose® CL-2B crosslinked agarose gel size fractionation column (Sigma-Aldrich) to separate the PPS 14:DNI matrix vaccine composition based on particle size. Fractionation was carried out using 10 mM phosphate buffer containing 150 mM NaCl.

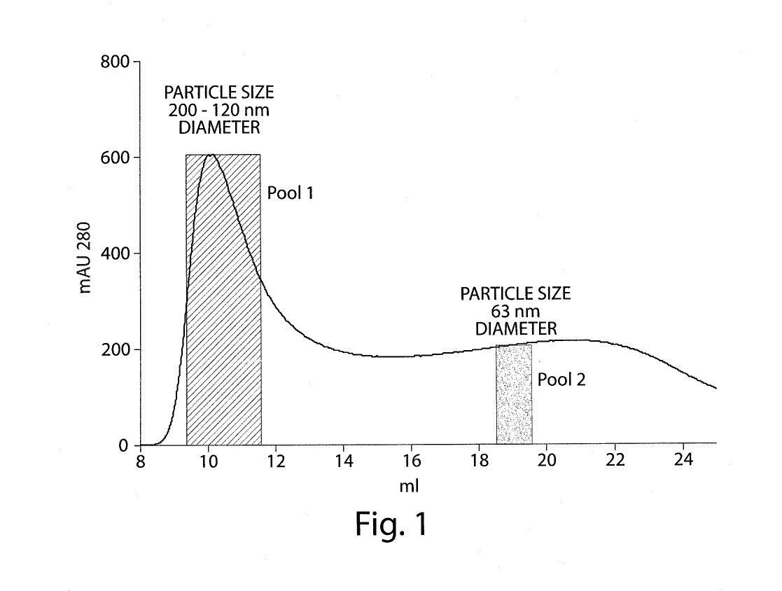

Two pools of PPS 14:DNI matrix vaccine particles were isolated for further evaluation. The first pool consisted of the 3 fractions (1 mL fractions) containing the void volume (pool 1) and a pool of two fractions (pool 2) that eluted from the column between the void volume and the position of the monomer DNI protein (83 kD) (see, FIG. 1). DNI elutes about the 24 mL position in FIG. 1. Pool 1 and Pool 2 were investigated further by refractive index, multi-angle laser light scattering chromatography (SEC-MALS-RI). In Pool 1, the particle size ranged from 120 to 200 nm in diameter; in Pool 2, the mean particle size was 63 nm in diameter. The composition of the pools is shown in Table 1:

TABLE 1
Composition of Pools 1 and 2, PPS 14:DNI matrix vaccine particles

| | | | | | PPS 14 (μg) |
|---|---|---|---|---|---|
| | DNI | PPS 14 | ratio DNI/PPS | PPS 14 (μg) in | in 5 μg DNI |
| Pool | (μg/μl) | (μg/μl) | 14 | 2 μg DNI dose | dose |
| 1 | 0.32 | 0.15 | 2.10 | 0.95 | 2.40 |
| 2 | 0.21 | 0.21 | 1.00 | 2.00 | 5.00 |

The larger particles of DNI in Pool 1 contained much less antigen (PPS 14) than the smaller particles of Pool 2. The particles in Pool 2 consisted of 86% PPS 14 and 14% DNI protein as determined by the MALS software (Astra, Wyatt Technologies) (data not shown). Compositions were tested to confirm entrapment of the PPS 14 antigen by the crosslinking reaction. Five compositions were prepared and subjected to SDS-PAGE:

### Compositions

1. Pool 1 (2.4 μg PPS 14:DNI, particle size 200-120 nm)
2. Pool 2 (5.0 μg PPS 14:DNI, mean particle size 63 nm)
3. Whole PCMV reaction mixture (crosslinked, 0.25% glutaraldehyde, non-fractionated)
4. Whole PCMV reaction mixture (crosslinked, 0.05% glutaraldehyde, non-fractionated)
5. Control: DNI only (uncrosslinked) from which Pool 1 and Pool 2

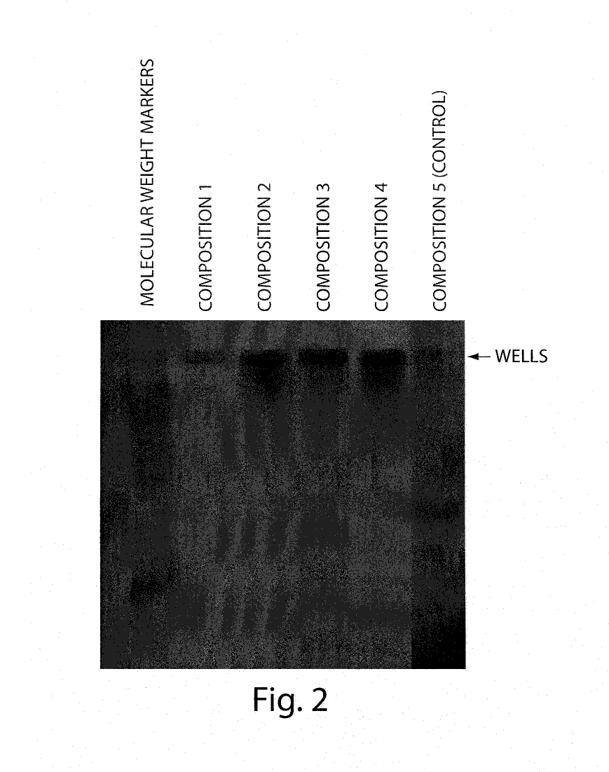

As shown in FIG. 2, compositions 1-4 all showed extensive crosslinking of the DNI carrier protein as evidenced by the shift of bands to higher molecular weight species on SDS-PAGE. Composition 4 (whole PCMV reaction mixture crosslinked with 0.05% glutaraldehyde) showed crosslinking of DNI but demonstrated a wider range of bands ranging from lower molecular weight species up to higher molecular weight bands (cf. lanes for Compositions 1, 2, 3).

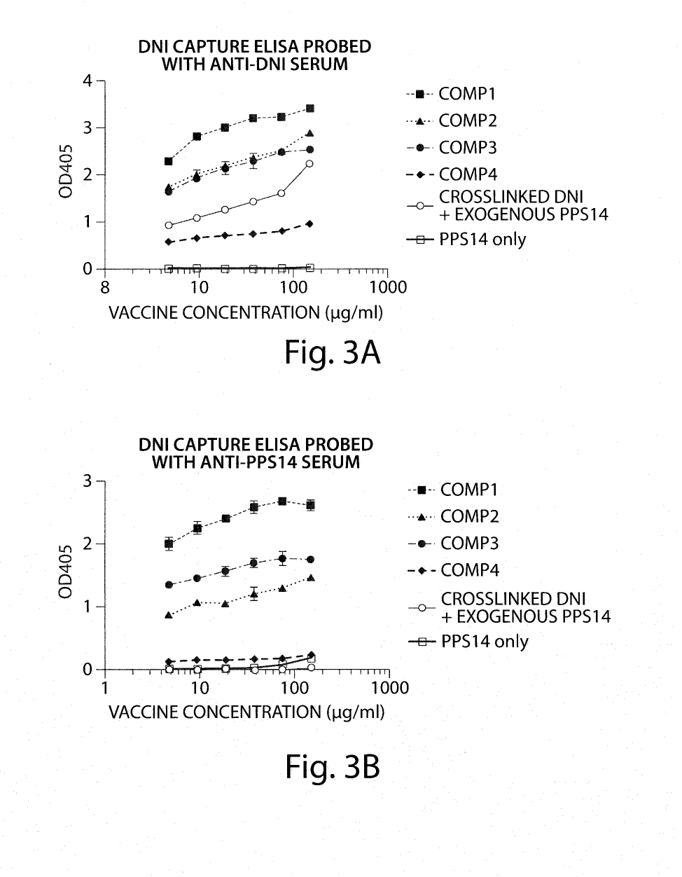

To confirm that the PPS 14 antigen remained associated with the crosslinked DNI matrix, a DNI capture ELISA was performed in which the PPS 14:DNI vaccine formulations were allowed to bind to immobilized mouse anti-DNI capture antibody (made in-house) for 2 hours at room temperature. Unbound material was washed away with PBS-0.5% Tween-20 (PBST) and rabbit anti-PPS 14 antibody (Miravista Diagnostics) was used to detect polysaccharide that remained associated with the captured DNI matrix protein. Immobilization of DNI matrix compositions was confirmed using a rabbit anti-DNI antibody (gift from John Collier, Harvard Medical School). Rabbit anti-PPS 14 or rabbit anti-DNI antibody was detected by incubation with monoclonal anti-rabbit antibody conjugated to alkaline phosphatase (Sigma) and visualized by addition of p-nitrophenyl phosphate substrate. In control experiments, a composition of PPS 14 only (not associated with DNI) and a composition of crosslinked DNI (without polysaccharide antigen) to which exogenous PPS 14 was added were run simultaneously in the assay. In the final detection step no PPS 14 was observed in these control groups (see, FIG. 3B). In contrast, when the anti-DNI capture antibody was incubated with the PCMV compositions 1-4, the PPS 14 within the DNI matrix was detectable by the PPS 14-specific detection antibody. This confirms that the PPS 14 antigen remains associated with the crosslinked DNI matrix.

Detection signal (OD 405) at increasing concentration of test composition are plotted in FIGS. 3A (anti-DNI detection) and 3B (anti-PPS 14 detection). Referring to FIG. 3B, the PCMV prepared with 0.05% glutaraldehyde (Composition 4) showed a weaker detection signal with the anti-PPS 14 detection antibody, which may correspond to the wider range of molecular species sizes on the SDS-PAGE gel. The Pool 1 and Pool 2 compositions (Compositions 1 and 2) and the whole PCMV reaction mixture crosslinked with 0.25% glutaraldehyde (Composition 3) showed migration with higher molecular weight species on the SDS-PAGE gel (FIG. 2). These samples (Compositions 1-3) also showed the highest detection of PPS 14 in the capture ELISA (FIG. 3B). Immunogenicity of the PCMV formulations was tested:

### Inoculum Compositions

1. Pool 1+alum (2.4 μg PPS 14)

2. Pool 2+alum (5.0 μg PPS 14)

3. Whole PCMV (0.25% glutaraldehyde)+alum

4. Whole PCMV (0.05% glutaraldehyde)+alum

5. Control: 5 μg PPS 14 antigen alone (no alum)

The PCMV inoculum compositions including an alum adjuvant (170 μg alum per dose) were injected (5 μg DNI) in a 100 μL volume by intraperitoneal route into mice using the following dosing regimen (see Table 2, below). A control group of mice was also immunized with 5 μg PPS 14 antigen alone. A group of naïve, unvaccinated mice was also included as a control group.

TABLE 2
PPS 14:DNI matrix vaccine composition dosing and
sampling schedule

| Day | Activity |
|---|---|
| −1 | pre-bleed |
| 0 | immunization #1 |
| 10 | blood sample #1 |
| 14 | immunization #2 |
| 24 | blood sample #2 |
| 28 | immunization #3 |
| 38 | blood sample #3 |
| 55 | blood sample #4 |

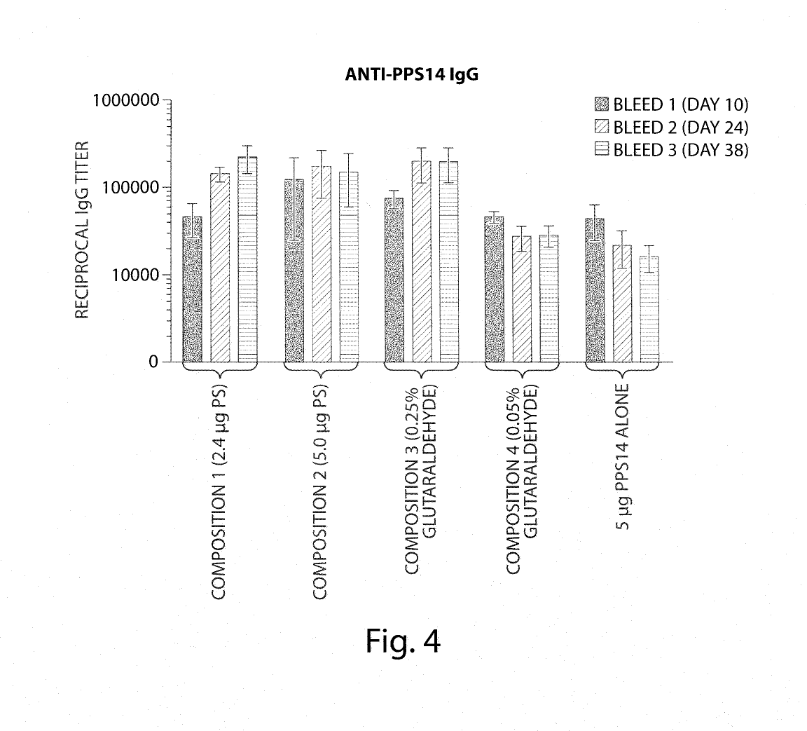

Serum anti-PPS 14-specific IgG responses were assayed by PPS 14 ELISA and plotted as individual titers and endpoint geometric mean titer (GMT). As seen in FIG. 4, inoculum composition 1 (alum adjuvanted Pool 1 fractions at 2.4 μg PS), having PPS 14:DNI PCMV particle sizes ranging from 200 nm to 120 nm in diameter, were surprisingly found to be as immunogenic or superior to inoculum composition 2 (alum adjuvanted Pool 2 fractions at 5.0 μg PS), having PPS 14:DNI PCMV mean particle size of 63 nm in diameter. The comparable immunogenicity of the larger sized PCMV particles, at half the entrapped antigen dose compared to the smaller sized Pool 2 fractions, indicates that the size of the PCMV particle affects the immunogenicity or potency of the vaccine composition.

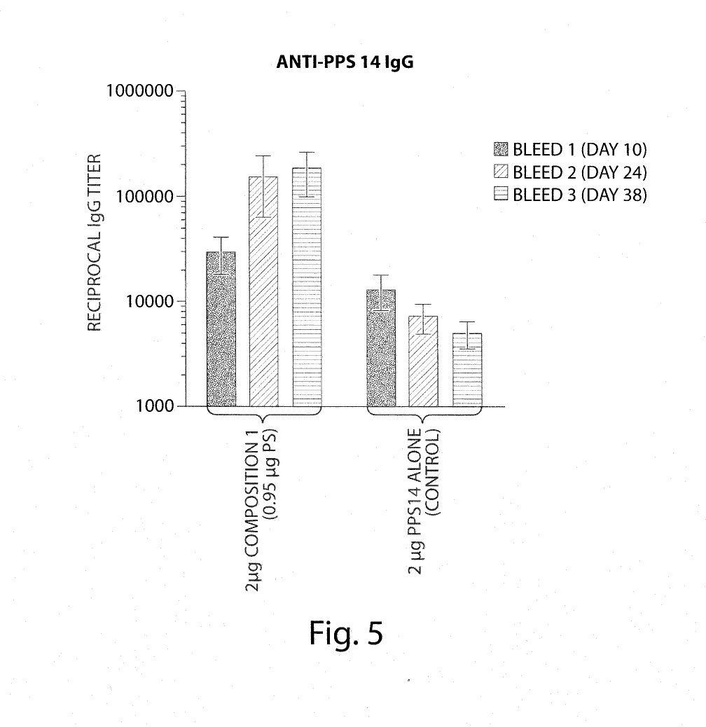

The experiment was repeated using 2 μg of alum adjuvanted Pool 1 (0.95 μg PPS 14), compared against 2 μg PPS 14 alone. As shown in FIG. 5, the anti-PPS 14 titers indicate that the larger particle size vaccine composition (i.e., Pool 1, with particle sizes ranging from 120 to 200 nm in diameter) showed superior immunogenicity even though the antigen dose was less than half of the antigen-only inoculum (0.95 μg PS vs. 2.0 μg PS).

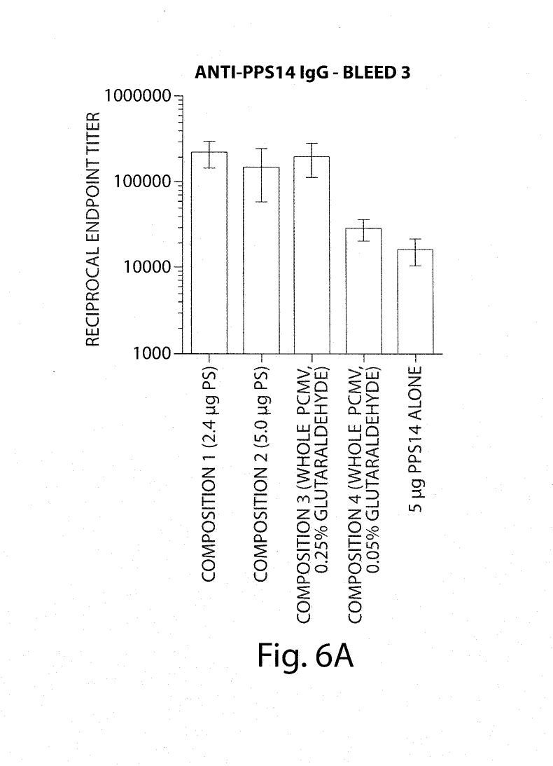

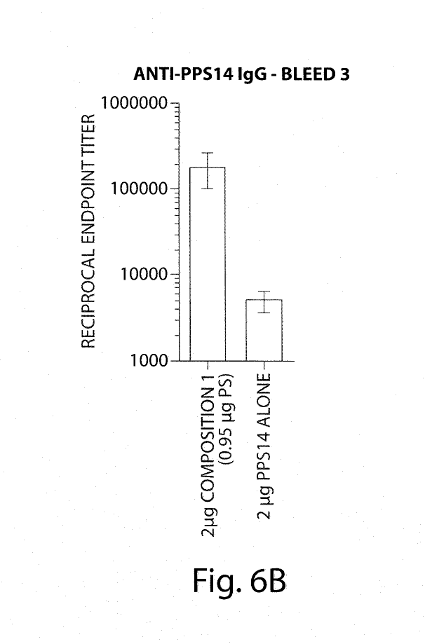

Endpoint geometric mean titers from the above immunizations were compared (Table 3, below) and plotted (FIG. 6).

TABLE 3
PPS 14 IgG Endpoint GMT Comparisons

| Dose of Polysaccharide (PS) Antigen in Inoculum | Day 38 GMT |
|---|---|
| Composition 1 (pool 1; 2.4 μg PS) | 182,127* |
| Composition 2 (pool 2; 5.0 μg PS) | 77,622 |
| Composition 3 (whole reaction mix; 0.25% | 138,026 |
| crosslinker; 5.0 μg PS) | |
| Composition 4 (whole reaction mix; 0.05% | 25,076 |
| crosslinker; 5.0 μg PS) | |
| Control: PPS 14 alone (5.0 μg PS) | 10,913 |
| Composition 1 (pool 1; 0.95 μg PS) | 135,148** |
| Control: PPS 14 alone (2.0 μg PS) | 1,061 |

*17-fold higher GMT compared to control at day 38 (3 immunizations at 0, 14, 28 days)
**127-fold higher GMT compared to control at day 38 (3 immunizations at 0, 14, 28 days)

The results indicate that the larger particle size vaccine compositions were much more immunogenic than the controls or the composition composed of the small particle size pool, even at a significantly reduced dose of antigen (e.g., 0.95 μg PS vs. 2.4 μg PS & 5.0 μg PS in the two experiments), further indicating that the particle size of the crosslinked carrier protein has a significant impact on the host immune response to the carried (entrapped) antigen.

### Example 2

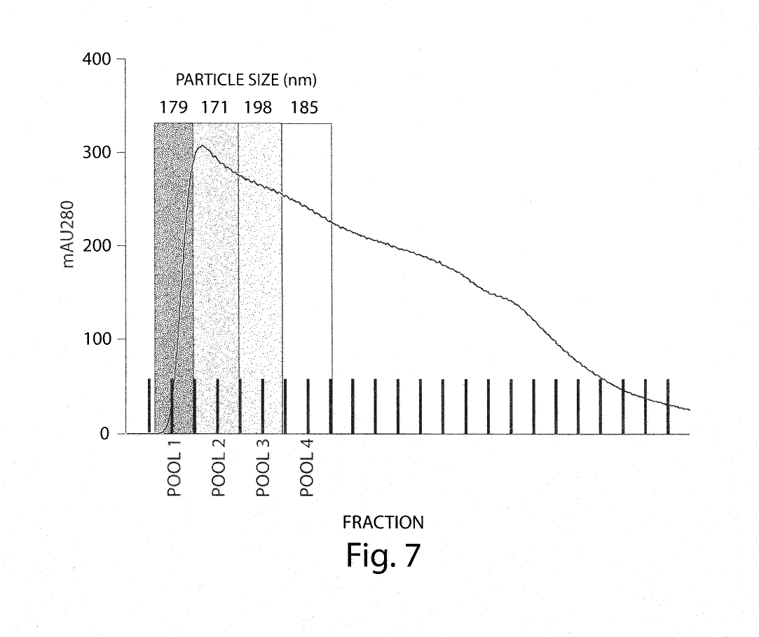

A matrix vaccine composition was prepared using as an antigen Salmonella typhi polysaccharide antigen Vi (extracted from Salmonella enterica serovar Typhi strain Ty2) and using as a carrier protein the dominant negative mutant (DNI) form of B. anthracis protective antigen (PA) expressed from Escherichia coli, to make Vi:DNI protein capsular matrix vaccine (Vi:DNI PCMV). The polysaccharide antigen (Vi) and carrier protein (DNI) were mixed at a 1:1 weight ratio and were present at 7.5 mg/ml for each component. Crosslinking of the DNI carrier protein was initiated by adding glutaraldehyde as a crosslinking agent to a final glutaraldehyde concentration of 0.25%. The crosslinking reaction was carried out in a total volume of 0.5 ml by incubating at 4° C. for 23 hours. At that time, sodium cyanoborohyride, which reduces Schiff bases, was added to a concentration of 20 mg/ml and the reaction mixture was incubated an additional hour. A portion of the reaction mixture was applied to a 25 ml Sepharose® CL-2B crosslinked agarose gel size fractionation column (Sigma-Aldrich) to separate the Vi:DNI matrix vaccine composition based on particle size. Fractionation was carried out using 10 mM phosphate buffer containing 150 mM NaCl. Four pools of Vi:DNI PCMV eluted fractions were isolated for further evaluation. (See, FIG. 7.)

The four pools were investigated further by dynamic light scattering (DLS). The particle size for each fraction pool is shown above the corresponding pool in FIG. 7. For Pool 1, the particle size was calculated at 179 nm. The particle size in Pool 2 was 171 nm in diameter. The particle size in Pool 3 was 198 nm in diameter, and the particle size in Pool 4 was calculated to be 185 nm in diameter. Dynamic light scattering provides the size of the largest components in the pooled fractions. It does not provide a size range of particles, nor does it provide a reading on the percentage of particles at the largest size.

Compositions comprising Pools 1-4 were used to immunize mice in accordance with the following protocol.

### Inoculum Compositions

1. Pool 1+alum

2. Pool 2+alum

3. Pool 3+alum

4. Pool 4+alum

5. Whole PCMV reaction mixture (unfractionated)+alum

6. Control: 10 μg Vi PS antigen alone (no alum)

The PCMV inoculum compositions including an alum adjuvant (170 μg alum per dose) were injected (10 μg by protein in a 100 μl volume by intraperitoneal route into mice using the following dosing regimen (see Table 4, below). A control group of mice was also immunized with 10 μg Vi polysaccharide (Vi PS) antigen alone. A group of naïve, unvaccinated mice was also included as a control group.

TABLE 4
Vi:DNI matrix vaccine composition dosing and sampling schedule

| Day | Activity |
|---|---|
| −1 | pre-bleed (blood sample #0) |
| 0 | immunization #1 |
| 10 | blood sample #1 |
| 14 | immunization #2 |
| 24 | blood sample #2 |
| 28 | immunization #3 |
| 38 | blood sample #3 |
| 55 | sacrifice, blood sample #4 |

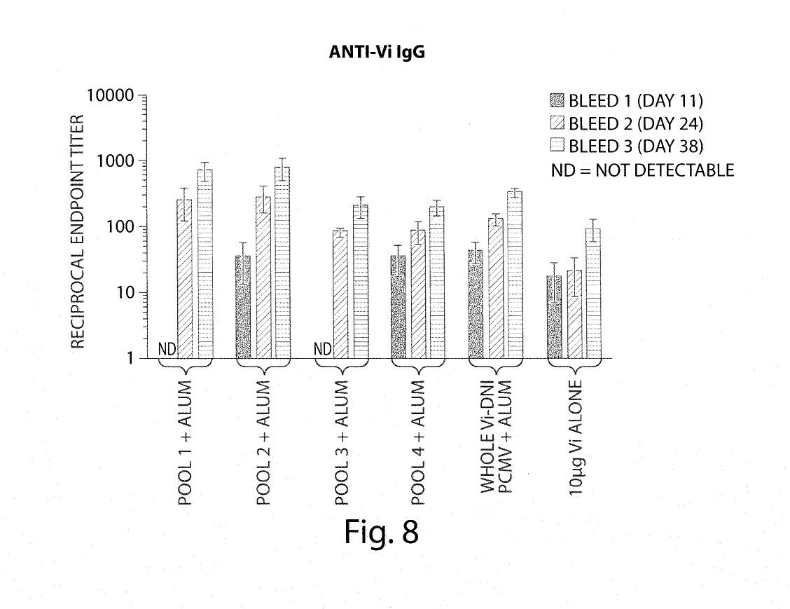

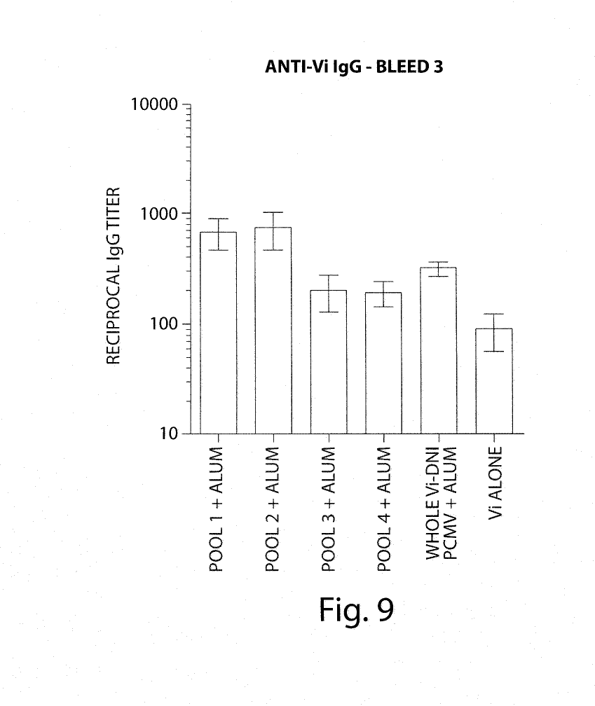

Serum anti-Vi PS-specific IgG responses were assayed by Vi ELISA and plotted as individual titers and endpoint GMT. Referring to FIGS. 8 and 9, sera from mice immunized with Composition 1 (Pool 1, alum adjuvant) and Composition 2 (Pool 2, alum adjuvant) Vi:DNI PCMVs indicated superior anti-Vi PS IgG immune responses as compared with sera from mice immunized with Composition 3 (Pool 3, alum adjuvant), Composition 4 (Pool 4, alum adjuvant), or Composition 5 (Whole unfractionated PCMV, alum adjuvant). FIG. 9 and Table 5 below show the superior geometric mean titers of mice immunized with Pool 1 or Pool 2 Vi:DNI PCMVs compared to mice immunized with Pool 3, Pool 4, and unfractionated Vi-DNI PCMV, from which the pools were taken.

TABLE 5
Vi:DNI IgG Endpoint GMT Comparisons

| Inoculum Composition (10 μg) | Day 38 GMT |
|---|---|
| Composition 1 (pool 1 + alum) | 504 |
| Composition 2 (pool 2 + alum) | 400 |
| Composition 3 (pool 3 + alum) | 168 |
| Composition 4 (pool 4 + alum) | 159 |
| Composition 5 (Whole Vi: DNI PCMV | 303 |
| (unfractionated) + alum) | |
| Control: 10 μg Vi PS alone | 40 |

Particle sizes are larger in pools 1 and 2 which may also entrap Vi polysaccharide more efficiently than smaller particles such as those in pools 3 and 4.

### Example 3

A further experiment on a size fractionated PPS 14:DNI protein capsular matrix vaccine was conducted, following the protocol of Example 1 but on a larger scale. A polysaccharide antigen (PPS 14) and carrier protein (DNI) were mixed at a 1:1 weight ratio and were present at 7.5 mg/ml for each component. Crosslinking of the DNI carrier protein was initiated by adding glutaraldehyde as a crosslinking agent to a final glutaraldehyde concentration of 0.25%. The crosslinking reaction was carried out in a total volume of 1.5 ml by incubating at 4° C. for 23 hours. At that time, sodium cyanoborohyride, which reduces Schiff bases, was added to a concentration of 20 mg/ml and the reaction mixture was incubated an additional hour.

A portion of the PPS 14:DNI PCMV reaction mixture was applied to a 100 ml Sepharose® CL-2B crosslinked agarose gel size fractionation column (Sigma-Aldrich) to separate the PPS 14:DNI matrix vaccine composition based on particle size. Fractionation was carried out using 10 mM phosphate buffer containing 150 mM NaCl.

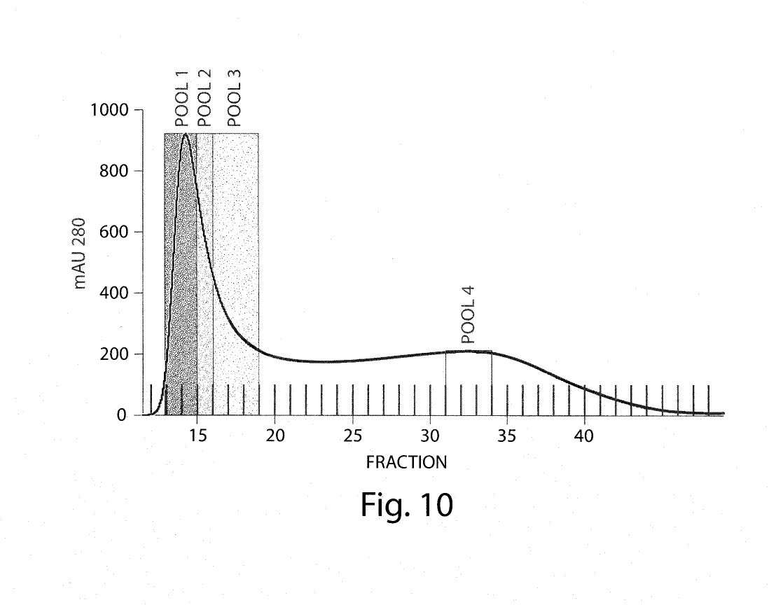

Four pools of PPS 14:DNI matrix vaccine particles were isolated for further evaluation. Referring to FIG. 10, collected fractions are indicated by short vertical lines along the x-axis. Pooling of fractions is indicated by shading.

The amount of PPS 14 antigen present in the fractions was determined using a phenol-sulfuric acid assay for carbohydrates. The amount of DNI present in the fractions was determined by UV₂₈₀ absorbance. The ratio of DNI to PPS 14 in the fractions was determined. The results are shown in Table 6.

TABLE 6
Composition of fractions from size fractionation of a
PPS 14:DNI matrix vaccine

| | DNI carrier | PPS 14 | Ratio of |
|---|---|---|---|
| | protein | antigen | carrier/ |
| Fraction | (mg/ml) | (mg/ml) | antigen |
| 13 | 0.07 | 0.007 | 10.0 |
| 14 | 0.59 | 0.038 | 15.5 |
| 15 | 0.89 | 0.052 | 17.1 |
| 16 | 0.63 | 0.070 | 9.0 |
| 17 | 0.39 | 0.076 | 5.1 |
| 18 | 0.28 | 0.076 | 3.7 |
| 19 | 0.24 | 0.076 | 3.2 |
| 20 | 0.22 | 0.118 | 1.9 |
| 21 | 0.18 | 0.135 | 1.3 |
| 22 | 0.20 | 0.158 | 1.3 |
| 23 | 0.22 | 0.178 | 1.2 |
| 24 | 0.17 | 0.202 | 0.8 |
| 25 | 0.19 | 0.227 | 0.8 |
| 26 | 0.21 | 0.247 | 0.9 |
| 27 | 0.18 | 0.260 | 0.7 |
| 28 | 0.19 | 0.240 | 0.8 |
| 29 | 0.20 | 0.216 | 0.9 |
| 30 | 0.20 | 0.250 | 0.8 |
| 31 | 0.22 | 0.227 | 1.0 |
| 32 | 0.21 | 0.225 | 0.9 |
| 33 | 0.19 | 0.189 | 1.0 |
| 34 | 0.20 | 0.167 | 1.2 |
| 35 | 0.20 | 0.135 | 1.5 |
| 36 | 0.17 | 0.081 | 2.1 |
| 37 | 0.30 | 0.148 | 2.0 |

Fractions were selected and pooled for further investigation as follows:
Pool 1—fractions 14, 15—DNI content 0.74 mg/ml
Pool 2—fraction 16—DNI content 0.63 mg/ml
Pool 3—fractions 17, 18, 19—DNI content 0.31 mg/ml
Pool 4—fractions 32, 33, 34—DNI content 0.20 mg/ml
The antigen and carrier protein composition of the pools is shown in Table 7:

TABLE 7
Composition of Pools 1-4, PPS 14:DNI matrix vaccine particles

| | | | | PPS 14 (μg) | PPS 14 (μg) |
|---|---|---|---|---|---|
| | DNI | PPS 14 | ratio DNI/ | in 0.5 μg | in 2 μg DNI |
| Pool | (μg/μl) | (μg/μl) | PPS 14 | DNI dose | dose |
| 1 | 0.74 | 0.045 | 16.4 | 0.03 | 0.12 |
| 2 | 0.63 | 0.070 | 9.0 | 0.06 | 0.22 |
| 3 | 0.31 | 0.076 | 4.1 | 0.13 | 0.52 |
| 4 | 0.20 | 0.194 | 1.0 | 0.48 | 1.91 |

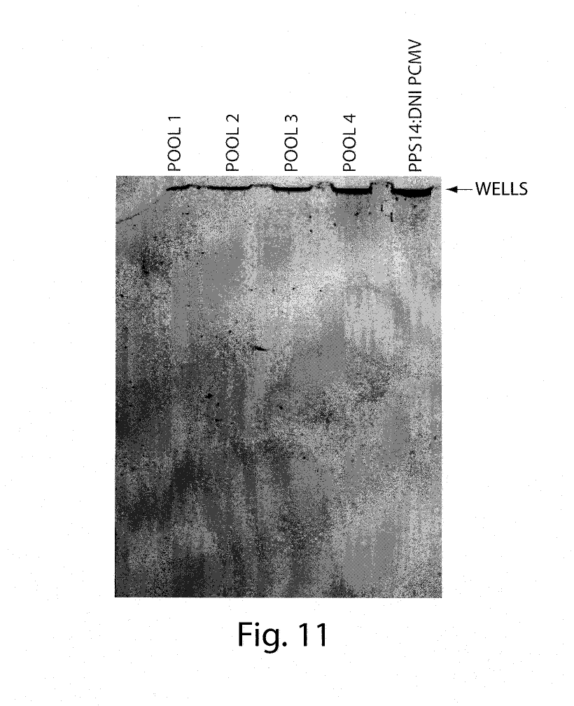

Crosslinking integrity of the PPS 14:DNI PCMV pooled fractions and the whole PCMV composition, from which the fractions were derived, was analyzed by SDS-PAGE (4-12% Bis-Tris gel) and Coomassie blue staining (see, FIG. 11). As shown in FIG. 11, the pooled fractions and the whole PCMV reaction all showed extensive crossinking of the DNI protein, as evidenced by the lack of migration into the stacking gel. The appearance of a smear below the well for Pool 4 similar to the smear below the well for the whole PPS 14:DNI PCMV composition indicates the presence of lower molecular weight species in these samples.

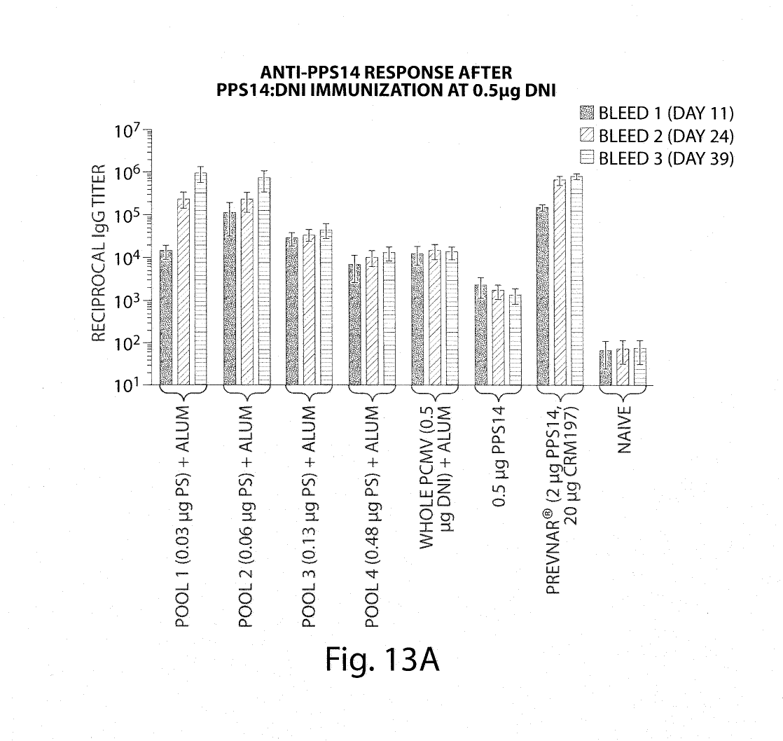

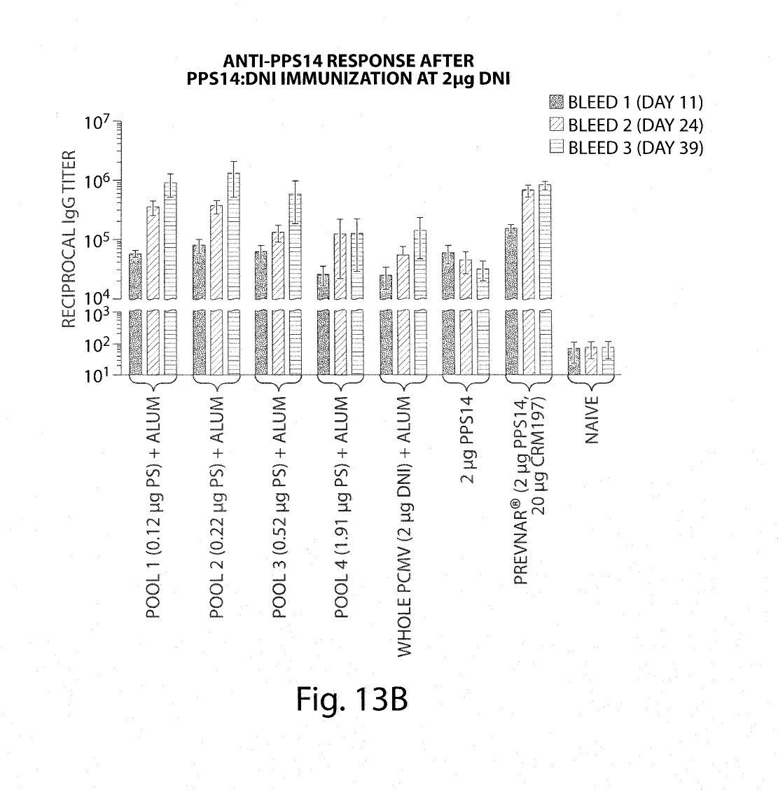

The PPS 14:DNI PCMV fraction pools and whole (unfractionated) PPS 14:PCMV matrix vaccine composition were also characterized using DNI capture ELISA probed with anti-PPS 14 serum to determine if the PPS 14 antigen remains entrapped and surface exposed (see, FIG. 13). Briefly, the vaccine formulations were allowed to bind to mouse anti-DNI capture antibody immobilized on a solid support. Unbound material was washed away and polyclonal rabbit anti-PPS 14 antibody (Miravista Diagnostics) was used to detect PPS 14 antigen that remained associated with the DNI matrix protein. A rabbit anti-DNI detection antibody was used to demonstrate that the matrix vaccine formulations were in fact captured by the DNI capture antibody.

PPS 14 was detected in all PCMV fractionation Pools 1-4. Interestingly, less PPS 14 antigen was detected in Pool 4, suggesting there was less entrapment of PPS 14. This result is consistent with the SDS-PAGE gel which showed evidence of lower molecular weight species in Pool 4. At the concentration used in the capture ELISA, the PPS 14 antigen signal for the whole PCMV composition was faint, however the presence of PPS 14 in the carrier matrix was clearly detected when a higher concentration of whole (unfractionated) PCMV composition was incubated with the capture DNI antibody (data not shown). In contrast, when crosslinked DNI with exogenously added PPS 14 was incubated with the capture DNI antibody, there was no detection by the PPS 14 antibody, indicating lack of entrapment of exogenous PPS 14 by crosslinked DNI (FIG. 13A, open squares (□)). Pools 1-4 and the crosslinked DNI control were bound by the capture DNI antibody (FIG. 13B). The whole PCMV composition was also bound by the DNI capture antibody and detected with DNI detection antibody when higher concentrations of whole PCMV composition were incubated with the capture antibody (data not shown). Therefore, the DNI capture ELISA demonstrated that there was significant entrapment and surface localization of PPS 14 within the DNI protein matrix.

Fractions comprising Pools 1-4 from the experiment were used to immunize mice in accordance with the following procedure. Compositions were prepared from the pooled fractions and whole, unfractionated PPS 14:DNI PCMV including alum adjuvant were prepared for immunization studies.

### Inoculum Compositions

Groups of 5-6 mice each (80 mice total) were innoculated with an inoculum composition according to the following design:

Group 1—0.5 μg Pool 1 (left void)+alum (6)

Group 2—2 μg Pool 1 (left void)+alum (6)

Group 3—0.5 μg Pool 2 (mid void)+alum (6)

Group 4—2 μg Pool 2 (mid void)+alum (6)

Group 5—0.5 μg Pool 3 (right void)+alum (6)

Group 6—2 μg Pool 3 (right void)+alum (6)

Group 7—0.5 μg Pool 4 (trailing peak)+alum (6)

Group 8—2 μg Pool 4 (trailing peak)+alum (5)

Group 9—0.5 μg Whole PCMV composition+alum (6)

Group 10—2 μg Whole PCMV composition+alum (5)

Group 11—positive control: 0.5 μg PPS 14 antigen alone (5)

Group 12—positive control: 2 μg PPS 14 antigen alone (6)

Group 13—comparative control: Prevnar® (commercial pneumococcal heptavalent conjugate vaccine) (5)

Group 14—negative control: Naive, unvaccinated (5)

The dosages above are listed by carrier protein (DNI) amount.

Prevnar® pneumonia vaccine, manufactured and marketed by Wyeth (Madison, N.J., USA), is an alum-adjuvanted conventional conjugate vaccine that contains 2 μg of PPS 14 along with six other S. pneumoniae polysaccharide antigens, all crosslinked with a total 20 μg CRM197 as a carrier protein. By using Prevnar® vaccine as a control vaccine, the immune responses to PPS 14 elicited by size-fractionated protein capsular matrix vaccines (PCMVs) was directly compared to the PPS 14-specific response elicited by a conventional conjugate vaccine. A group of naïve mice was also included as a control group.

The immunization schedule is set forth in Table 8:

TABLE 8
PPS 14:DNI vaccine composition dosing and sampling schedule

| Day | Activity |
|---|---|
| −1 | pre-bleed (blood sample #0) |
| 0 | immunization #1 |
| 10 | blood sample #1 |
| 13 | immunization #2 |
| 24 | blood sample #2 |
| 27 | immunization #3 |
| 38 | blood sample #3 |
| 55 | sacrifice, blood sample #4 |

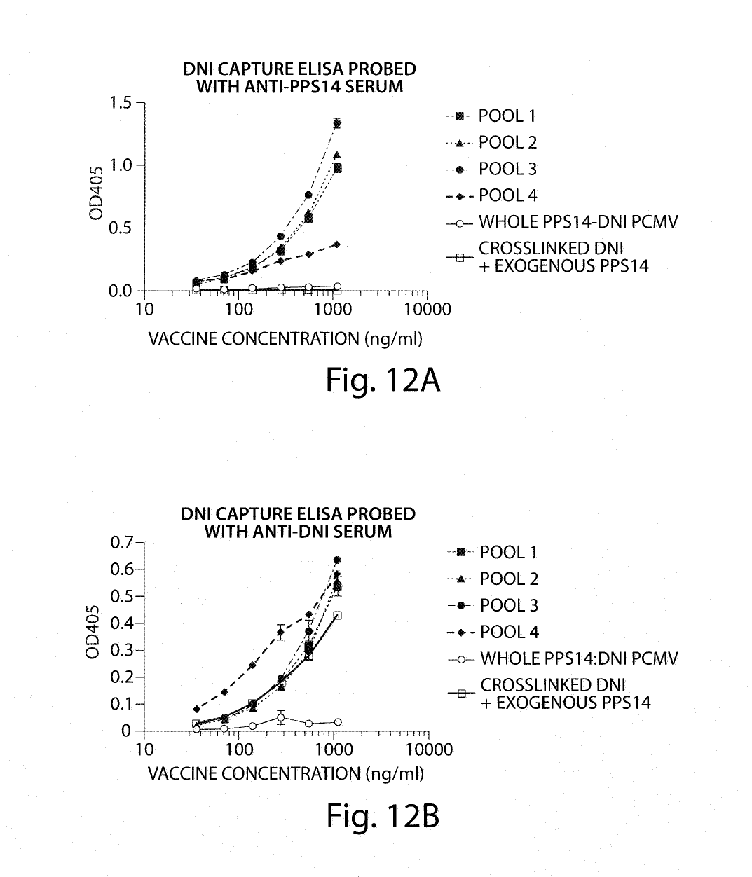

Serum anti-PPS 14-specific IgG responses were assayed by PPS 14 ELISA and plotted as individual titers and endpoint GMT. (See, FIG. 12.)

Referring to FIG. 13A, sera from mice immunized with Pools 1-4 or the whole PCMV exhibited markedly higher PPS 14-specific IgG responses than sera from mice immunized with PPS 14 alone at 0.5 μg, particularly Pools 1 and 2, which were determined to contain 0.03 μg PS and 0.06 μg PS antigen, respectively. FIG. 13A shows the anti-PPS 14-specific IgG response following immunization with Pools 1-4 and whole PCMV with 0.5 μg DNI; FIG. 13B shows the anti-PPS 14-specific IgG response following immunization with Pools 1-4 and whole PCMV with 2.0 μg DNI. PPS 14-specific IgG titers increased over time in sera from mice immunized with Pools 1-4 or whole PCMV relative to sera from mice immunized with 0.5 μg PPS 14 alone.

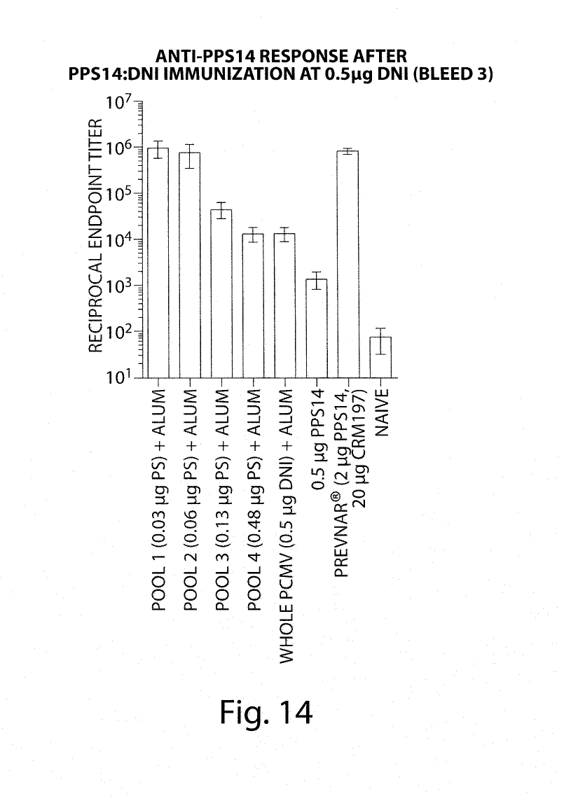

FIG. 14 illustrates anti-PPS 14 endpoint titers at Day 38 (blood sample #3) for the immunizations at 0.5 μg DNI, and the Day 38 geometric mean titers are shown in Table 9, below.

TABLE 9
PPS 14:DNI Immunization at 0.5 μg DNI; Anti-PPS 14
Endpoint Titers

| Inoculum Composition (amount of PPS 14 antigen) | Day 38 GMT |
|---|---|
| Pool 1 + alum (0.03 μg PPS 14) | 501,103 |
| Pool 2 + alum (0.06 μg PPS 14) | 228,543 |
| Pool 3 + alum (0.13 μg PPS 14) | 27,691 |
| Pool 4 + alum (0.48 μg PPS 14) | 4,615 |
| Whole PPS 14: DNI PCMV (unfractionated) + alum | 9,413 |
| (0.5 μg DNI) | |
| Control: 0.5 μg PPS 14 antigen alone | 800 |
| Prevnar ® (2 μg PPS 14, 20 μg CRM197) | 776,047 |
| Control: Naïve mice (unvaccinated) | 50 |

It is significant to note the anti-PPS 14 antigen titers achieved by immunization with the matrix vaccine fractions compared to immunization using the antigen alone. The results confirm the advantage in terms of immunogenicity achieved by entrapping antigen in a crosslinked protein carrier.

Fractions having the largest crosslinked DNI particles (Pools 1-3) showed significantly greater immunogenicity than antigen alone or even Pool 4 (characterized by lower molecular weight DNI carrier protein particles compared to Pools 1-3) or whole PPS 14:DNI vaccine composition (containing all particle size ranges). These results are especially surprising when it is considered that the entrapped antigen content of the Pool 1, 2 and 3 compositions was 3-17 times lower than the other PCMV compositions (Pool 4 and whole PCMV) and the PS antigen-only control.

In comparison to the conventional Prevnar® vaccine, Pools 1 and 2, which contained larger sized carrier protein particles, elicited comparable anti-PPS 14 responses. This comparable anti-PPS 14 response is remarkable given that the actual dose of PPS 14 antigen administered for PCMV Pools 1 and 2 was significantly less than the dose of PPS 14 contained in Prevnar®: around 66-fold less PPS 14 antigen in the Pool 1 dose compared to the Prevnar® dose, and around 33-fold less PPS 14 antigen for the Pool 2 dose compared to the Prevnar® dose.

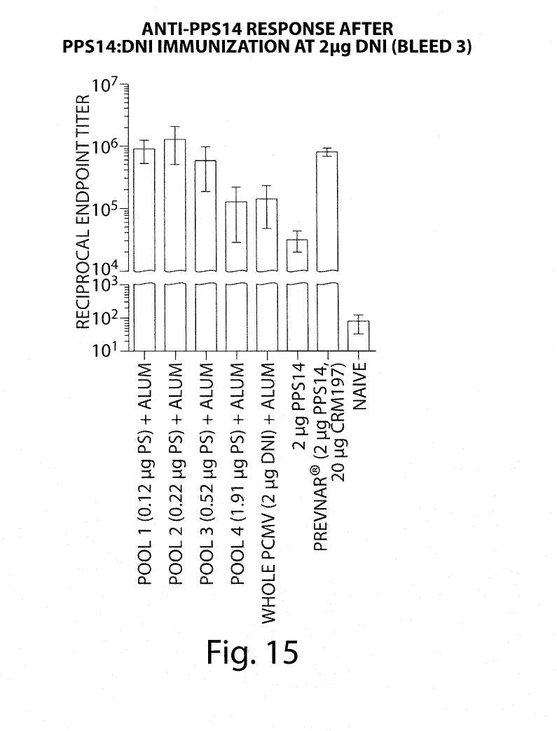

FIG. 15 illustrates anti-PPS 14 endpoint titers at Day 38 (blood sample #3) for the immunizations at 2 μg DNI, and the Day 38 geometric mean titers are shown in Table 10, below.

TABLE 10
PPS 14:DNI Immunization at 2 μg DNI; Anti-PPS 14 Endpoint Titers

| Inoculum Composition (amount of PPS 14 antigen) | Day 38 GMT |
|---|---|
| Pool 1 + alum (0.12 μg PPS 14) | 619,077 |
| Pool 2 + alum (0.22 μg PPS 14) | 586,094 |
| Pool 3 + alum (0.52 μg PPS 14) | 256,531 |
| Pool 4 + alum (1.91 μg PPS 14) | 36,933 |
| Whole PPS 14: DNI PCMV (unfractionated) + alum | 13,846 |
| (2 μg DNI) | |
| Control: 2 μg PPS 14 antigen alone | 12,335 |
| Prevnar ® (2 μg PPS 14, 20 μg CRM197) | 776,047 |
| Control: Naïve mice (unvaccinated) | 50 |

Again, the anti-PPS 14 antigen titers achieved by immunization with the matrix vaccine fractions were superior to those achieve by immunization using the antigen alone. The results confirm the advantage in terms of immunogenicity achieved by entrapping antigen in a crosslinked protein carrier.

Fractions having the largest crosslinked DNI particles (Pools 1-3, see FIG. 11) showed significantly greater immunogenicity than antigen alone or even Pool 4 (characterized by lower molecular weight DNI carrier protein particles compared to Pools 1-3) or whole PPS 14:DNI vaccine composition (containing all particle size ranges). These results are especially surprising when it is considered that the entrapped antigen content of the Pool 1, 2 and 3 compositions was 3.7-16.7 times lower than the other PCMV compositions (Pool 4 and whole PCMV) and the PS antigen-only control.

Pools 1, 2 and 3, which contained larger sized crosslinked DNI carrier particles, elicited anti-PPS 14 responses on the same order as with the conventional Prevnar® vaccine. This comparable anti-PPS 14 response is remarkable, given that the actual dose of PPS 14 antigen administered in PCMV Pools 1, 2 and 3 was significantly less than the dose of PPS 14 contained in the Prevnar® injections: around 17-fold less PPS 14 for Pool 1, around 9-fold less PPS 14 for Pool 2, and around 4-fold less PPS 14 for Pool 3.

Referring again to FIG. 15, collectively PPS 14-specific IgG titers increase in mice immunized with PCMV fractionated pools, unfractionated PCMV composition, or Prevnar® compared to sera from mice immunized with PPS 14 alone. Mice immunized with PPS 14 antigen alone show PPS 14-specific IgG titers decreasing over time at both 0.5 μg and 2 μg PPS 14 dosage levels. This suggests that “immunological memory” responses were elicited by the PCMV and the Prevnar® inocula.

These data indicate that presentation of capsular antigen as part of a matrix vaccine is not only more efficient than immunization with antigen alone but can be more efficient than conventional conjugate vaccines. This has important implications for vaccine formulation processes, indicating that judicious regulation of matrix particle size can dramatically simplify the vaccine design and production process and can markedly reduce the amount of antigen required to elicit a protective immune response.

It is evident that permitting the carrier protein crosslinking reaction in presence of desired antigen to continue to produce large matrix particles (i.e., >100 nm in diameter) entraps the antigen very efficiently. Also, production of larger crosslinked carrier particle sizes (or selecting a high molecular weight fraction from the reaction) substantially enhances the immunogenicity of the PCMV composition, even though when the particles contain very low amounts of antigen. The data show that size fractionation of the PCMV composition and immunizing animals with the larger size particles can induce enhanced anti-PS-specific IgG responses that are comparable to the responses induced by conventional conjugate vaccines. Moreover, these data indicate that memory immune responses elicited by conventional conjugate vaccines (e.g., Prevnar®), can also be obtained by immunization with a PCMV.

These Prevnar® controlled, PCMV particle sized data indicate that optimization of PCMV particle size and optimization of the amount of polysaccharide antigen entrapped and presented by the PCMV composition might lead to further enhancement of specific anti-PS antigen response to potentially eclipse the immune response elicited by such conventional vaccines as Prevnar®. Increasing the ratio of carried antigen to carrier protein in the final vaccine composition may be accomplished by adjusting the ratio between antigen polysaccharide and carrier protein prior to performing the carrier protein crosslinking reaction. Fractionation of the polysaccharides themselves before incorporation into PCMV matrices may also increase the immune responses obtained.

### Example 4

A matrix vaccine composition using Citrobacter freundii polysaccharide Vi and DNI carrier protein to produce a Vi:DNI PCMV. The polysaccharide (Citrobacter freundii Vi) and carrier protein (DNI) were mixed at a 1:1 weight ratio and were present at 7.5 mg/ml for each component. The crosslinking reaction was performed at 1.5 ml volume, glutaraldehyde being added as a crosslinking agent to a final concentration of 0.25%, and the reaction mixture incubated at 4° C. for 23 hours. At this point, sodium cyanoborohyride, which reduces Schiff bases, was added to a concentration of 20 mg/ml and the reaction mixture incubated an additional hour.

A conjugate vaccine was prepared as a comparative control using 0.9 mg/ml Vi antigen conjugated to bovine serum albumin.

A portion of the Vi:DNI PCMVreaction mixture was applied to a 100 ml

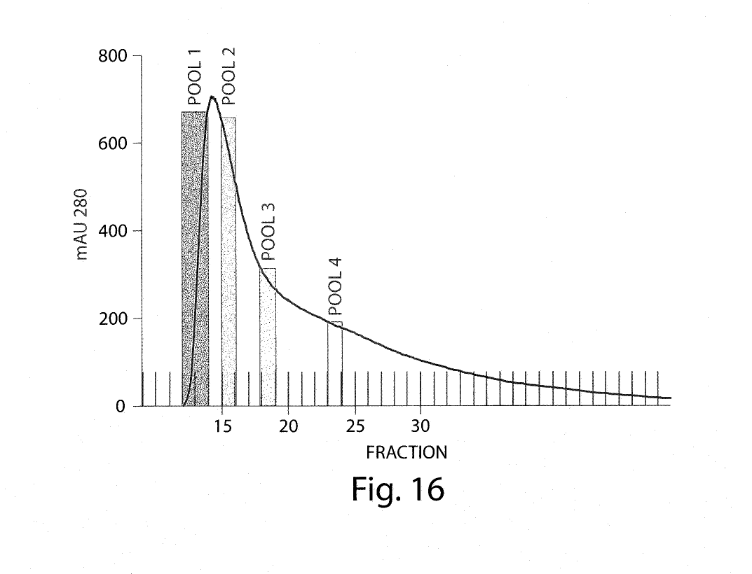

Sepharose® CL-2B crosslinked agarose gel size fractionation column (Sigma-Aldrich) to separate the Vi:DNI matrix vaccine composition based on particle size. Fractionation was carried out using 10 mM phosphate buffer containing 150 mM NaCl. Four pools of Vi:DNI PCMV eluted fractions were isolated for further evaluation. (See, FIG. 16.)

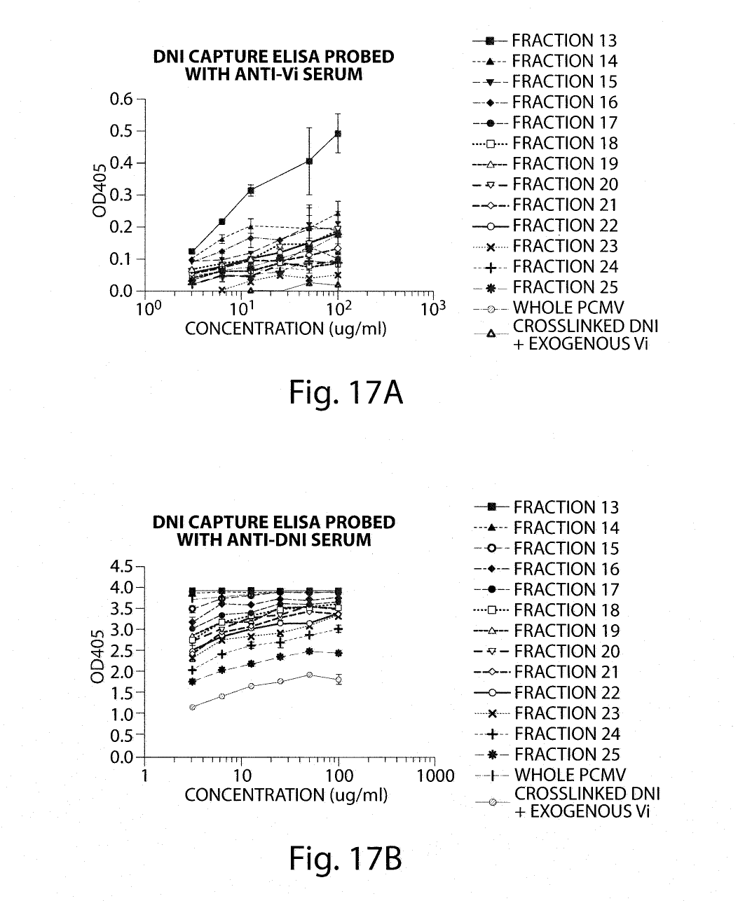

The individual fractions (FIG. 16) were evaluated by DNI capture ELISA (FIG. 17) and the results determined how the fractions were eventually pooled for making immunization compositions. Vi was detected in all PCMV fractionated formulations (FIG. 17A, Fractions 13-25) and in whole, unfractionated PCMV after capture by immobilized anti-DNI antibody. Vi was most strongly detected in Fraction 13, corresponding to the largest size particles that eluted from the column. Detection of Vi was essentially equivalent for the remaining fractions that were tested, with the general trend being that the earlier fractions containing larger size crosslinked DNI particles had slightly more detectable, surface-presented Vi antigen than the later fractions containing smaller DNI particles. This is presumably due to the smaller size particle entrapping less Vi. Vi PS was also detected in the whole PCMV reaction mixture from which these fractions were derived. In contrast, when cross-linked DNI with exogenously added Vi PS was incubated with the capture anti-DNI antibody, there was no detection by the Vi-specific antibody due to lack of entrapment of Vi. The individual Vi:DNI fractions, the unfractionated Vi:DNI, and the cross-linked DNI control were all bound by the capture anti-DNI antibody to a similar degree (FIG. 17B). Therefore, the DNI capture ELISA demonstrated that there was a detectable level of entrapped, surface localized Vi in the Vi:DNI protein matrix.

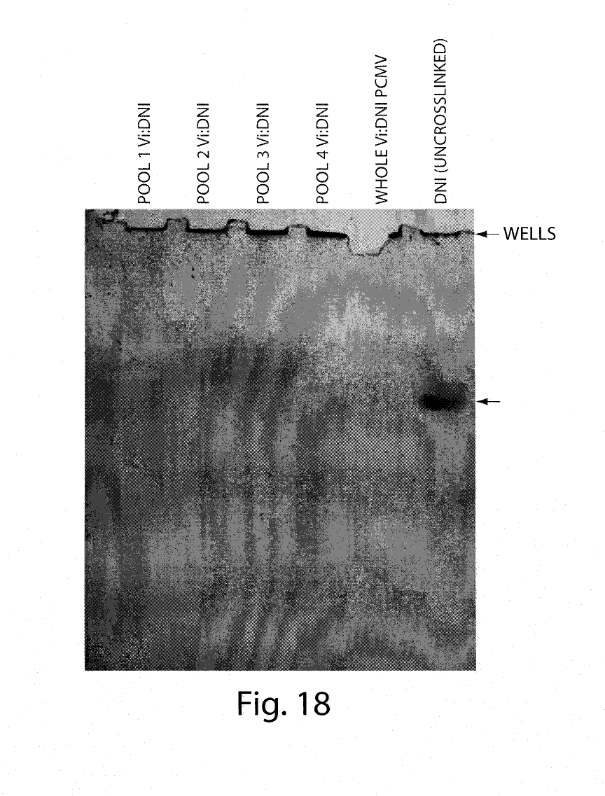

Fractions showing entrapped, presented Vi PS were pooled and used to prepare inoculum compositions (FIG. 17, shaded bars). Crosslinking integrity was analyzed by SDS-PAGE and Coomassie blue staining (FIG. 18). Pooled fractions and the whole PCMV reaction mixture contained very high molecular weight crosslinked DNI species that did not visibly migrate into the stacking gel but instead remained in the loading wells. Uncrosslinked DNI formed a lower molecular weight band (lower arrow). The amount of DNI present in the fractions was determined by UV₂₈₀ absorbance. The carrier/antigen ratio was estimated based on ratios of PS14:DNI PCMVs and the amount of Vi antigen present in a 10 μg dose based on DNI was calculated as set forth in Table 11:

TABLE 11
Composition of Pools 1-4, Vi:DNI matrix vaccine particles

| | | | Vi PS (μg) in |
|---|---|---|---|
| | DNI | ratio DNI/PS | 10 μg DNI dose |
| Pool | (μg/μl) | (estimated) | (estimated) |
| 1 | 0.36 | 15 | 0.66 |
| 2 | 0.66 | 10 | 1.0 |
| 3 | 0.33 | 5 | 2.0 |
| 4 | 0.20 | 1 | 10 |

Pooled fractions and related controls were prepared for use in immunization experiments:

### Inoculum Compositions

1. Pool 1 (10 μg DNI)+alum

2. Pool 2 (10 μg DNI)+alum

3. Pool 3 (10 μg DNI)+alum

4. Pool 4 (10 μg DNI)+alum

5. 10 μg DNI/Whole PCMV+alum

6. 5 μg PS Vi-BSA conjugate+alum2 μg S. typhi-derived Vi antigen alone (control)

7. 2 μg Citrobacter freundii-derived Vi antigen alone (control)

Compositions were injected intraperitoneally into mice using the standard dosing regimen (three injections at bi-weekly intervals) shown in Table 12. The Vi-BSA conjugate vaccine comparative control contained 0.9 mg/ml Vi covalently bound to BSA. A group of naïve, unvaccinated mice were also included as a control.

TABLE 12
Vi:DNI vaccine composition dosing and sampling schedule

| Day | Activity |
|---|---|
| −1 | pre-bleed (blood sample #0) |
| 0 | immunization #1 |
| 8 | blood sample #1 |
| 13 | immunization #2 |
| 22 | blood sample #2 |
| 27 | immunization #3 |
| 41 | blood sample #3 |
| 55 | sacrifice, blood sample #4 |

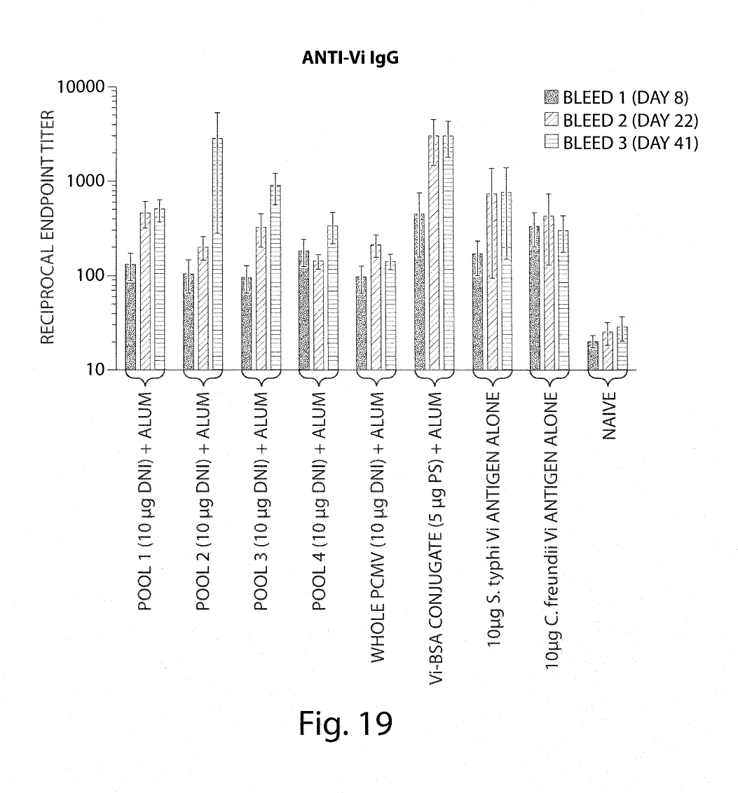

Serum anti-Vi PS-specific IgG responses were assayed by Vi ELISA and plotted as individual titers and endpoint GMT. (See, FIG. 19.)

FIG. 19 shows the kinetics of the anti-Vi-specific IgG response following immunization with 10 μg DNI for the fractionated PCMV pools or whole PCMV. Sera from mice immunized with the larger crosslinked DNI particles (Pools 1-3) developed higher Vi-specific IgG responses than sera from mice immunized with 10 μg Vi alone. In comparison, immunization with Pool 4 (smaller DNI particles) or whole PCMV generated Vi-specific antibody responses similar to when mice were immunized with Vi alone. Vi-specific IgG titers increased over time in sera from mice immunized with Pools 1-3 relative to sera from mice immunized with 10 μg Vi alone.

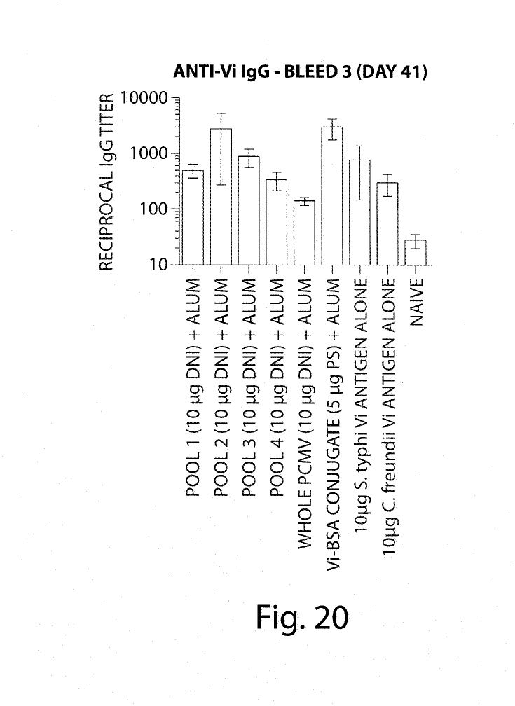

When the immunization regimen was completed, the Vi-specific IgG response at day 41 (FIG. 20), i.e., 2 weeks after the last immunization, was calculated as reciprocal geometric mean titers (GMTs), set forth in the table below.

TABLE 13
Vi:DNI Immunization at 10 μg DNI; Anti-PS Endpoint Titers

| Inoculum Composition (amount of Vi PS antigen) | Day 41 GMT |
|---|---|
| Pool 1 + alum (est. 0.66 μg PS antigen) | 400 |
| Pool 2 + alum (est. 1.0 μg PS antigen) | 528 |
| Pool 3 + alum (est. 2.0 μg PS antigen) | 606 |
| Pool 4 + alum (est. 10 μg PS antigen) | 264 |
| Whole Vi: DNI PCMV (unfractionated) + | 132 |
| alum (10 μg DNI) | |
| Control: 10 μg S. typhi Vi antigen alone | 200 |
| Control: 10 μg C. freundii Vi antigen alone | 230 |
| Vi-BSA conjugate (5 μg PS antigen) | 2263 |
| Control: Naïve mice (unvaccinated) | 25 |

Mice immunized with PCMV Pool 1, 2 or 3 developed anti-Vi-specific IgG GMT 2-3 fold higher than mice immunized with Vi alone. In contrast, immunization with the Vi-BSA conjugate induced 10-fold greater Vi-specific IgG GMT compared to immunization with Vi alone. The Vi conjugate and the Vi PCMV each induced anti-Vi antibody levels that were greater than Vi alone. The Vi-BSA conjugate (5 μg Vi) elicited higher titer anti-Vi antibodies than PCMV formulations containing less Vi antigen (see FIG. 16 and Table 11). With a less immunogenic polysaccharide (PS) such as Vi, compared to S. pneumoniae PPS 14, factors such as particle size and dosage can affect immunogenicity compared to immunizing with Vi PS alone. It was estimated, based on similar PS-protein amounts obtained from the PPS 14-DNI PCMV fractionation determinations since the elution profiles were similar), that the amount of Vi PS in size-fractionated PCMV Pool 1 (˜0.66 μg) was 13%, Pool 2 (˜1 μg) was 20%, and Pool 3 (˜5 μg) was 40% of the amount of Vi dose in the Vi-BSA conjugate. Thus, although the Vi conjugate elicited approximately 4-6 times the reciprocal anti-Vi antibody titer (2263) of Vi:DNI PCMV pools, if the response is normalized for dose then Vi:DNI PCMV Pool 1, Pool 2, and Pool 3 elicit reciprocal anti-Vi antibody titers of 3030, 2625, and 1515, respectively. Thus, the size-fractionated Vi:DNI PCMV compositions were comparable to slightly better than a Vi-protein conjugate.

Also, it is noted that the immunization of this example compared vaccine compositions based on different carrier proteins: BSA vs. a DNI matrix. Choice of carrier protein could have an effect on the immunopotency of the formulations. In addition, it was not determined if the Vi used to prepare the conjugate and PCMVs was the same, and the antigenicity and immunogenicity of Vi antigens from different sources can be distinctly different.

Collectively, for Vi antigen, the data of Examples 2 and 4 indicate: (i) that the PCMV formulated with higher concentrations of reactants where products are shifted to higher molecular weight species entraps polysaccharide more efficiently, (ii) that continuing the reaction to generate larger size particles (>120 nm diameter) enhances immunogenicity, and (iii) that size fractionation of the PCMV reaction and immunizing animals with the larger size particles induces comparable titers to a Vi/protein conjugate and higher titers thanVi antigen alone.

### Example 5

Mice from the immunization groups of Example 1 were maintained for an immunological memory experiment. Additional sera were collected at later time-points for seven months to monitor the kinetics of the anti-PPS 14 immune response. Ultimately, mice were boosted with homologous PCMV or PPS 14 formulations and sera were assayed for the development of a helper T cell-based memory (Tₕ-dependent) immune response.

Approximately 7 months after the three-dose immunization regimen of Example 1, mice were boosted with the compositions indicated in the table below:

TABLE 14
Immune Memory Response from Boost at Day 239

| | | | GMT four | GMT three |
|---|---|---|---|---|
| | | Pre-Boost | days after | weeks after |
| Original | | GMT | boost | boost |
| Immunizations | Boosted with | (Day 239) | (Day 243) | (Day 260) |
| Pool 1 + alum | 5 μg DNI/0.6 μg | 334,531 | 408,445 | 816,890 |
| (5 μg DNI/ | PS + alum | | | |
| 2.4 μg PS) | | | | |
| Pool 2 + alum | 5 μg DNI/0.6 μg | 152,691 | 266,251 | 863,756 |
| (5 μg DNI/ | PS + alum | | | |
| 5 μg PS) | | | | |
| 5 μg PS alone | 5 μg PS alone | 2,652 | 2,652 | 3,046 |
| 2 μg PS alone | 2 μg PS alone | 11,314 | 11,314 | 11,314 |
| Naïve | — | 119 | 100 | 71 |

Sera from boosted animals were collected at 4 days and 3 weeks post-boost to assay anti-PPS14 IgG immune responses. The Day 239 (4 days post-boost) time-point was chosen because, if memory immune responses were elicited, then a corresponding increase in specific IgG antibodies would be evident. The Day 260 (3 week post-boost) time-point was chosen because, if memory responses were elicited, then the IgG antibody titers would continue to rise. In general, PPS 14-specific IgG remained relatively high following the initial PPS 14:DNI PCMV immunization regimen, as indicated by the high pre-boost GMT ranging from 152,691 to 334,531 (Table 14, column 3). An increase in PPS 14-specific IgG antibodies was observed four days post-boost only in PCMV-immunized animals compared to mice immunized with PPS 14 antigen alone (Table 14, column 3 vs. column 4). Consistent with these data, the booster response increased significantly at the 3-week time-point in PCMV-immunized mice (GMT of 816,890 or 863,756), whereas PS only-immunized mice developed either no or minimal increase in anti-PPS 14 specific IgG (Table 14, column 5).

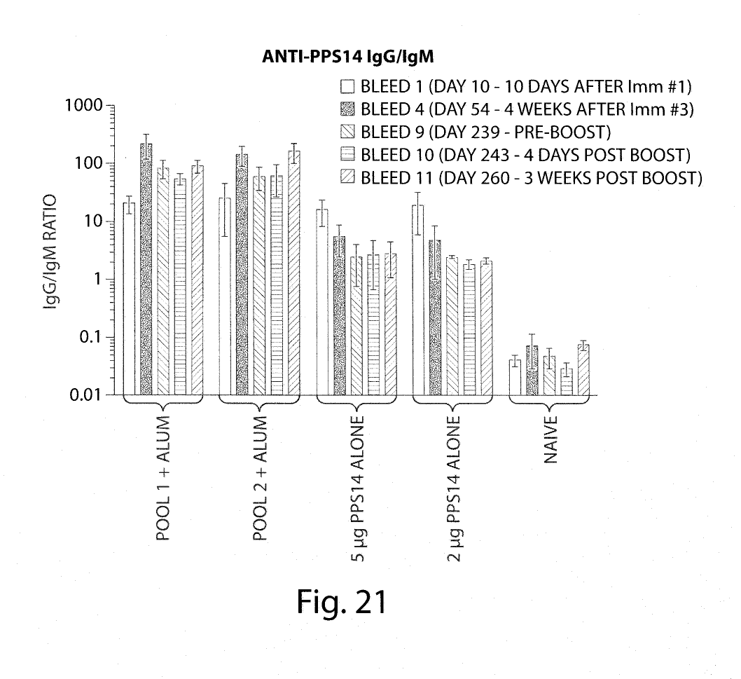

To further explore whether PCMV formulated PPS 14 elicits a Tₕ memory response, the ratio of IgG-to-IgM was assayed and determined (see, FIG. 21). Polysaccharide-only vaccines typically elicit IgM and low levels of IgG, whereas polysaccharide-protein conjugate vaccines elicit substantially higher levels of IgG. In general, IgM is more non-specific than IgG in its antigen binding affinity. Therefore a lower IgG-to-IgM ratio in naive animals was observed, indicating the presence of background levels of non-specific antibody in this group. Immunization with PPS 14 alone induced more of an anti-PPS 14-specific immune response compared to naive animals generating more specific IgG than IgM (IgG:IgM, ˜1:1). In sharp contrast, mice immunized with PPS 14:DNI PCMV Pool 1 and Pool 2 formulations elicited significantly more IgG compared to IgM, around a 10-100-fold change in ratio, compared to mice immunized with PS alone.

From these results, it is seen that PPS 14:DNI PCMV-immunized animals developed an increase in PPS 14-specific IgG after a booster immunization given about 7 months after an initial 3-dose immunization regimen. Sera collected 4 days and 3 weeks after this booster strongly indicate the development of a Tₕ-dependent, or “memory”, immune response. Moreover, the IgG:IgM ratio of mice immunized with PPS 14:DNI PCMV formulations (FIG. 21) further supports the observation of an elicited memory response.

All patents, patent applications, patent application publications, and other publications cited or referred to herein are incorporated by reference to the same extent as if each independent patent, patent application, patent application publication or publication was specifically and individually indicated to be incorporated by reference.

1. An immunogenic composition comprising (1) an antigen of interest and (2) a carrier protein, wherein said carrier protein is crosslinked to form a protein matrix, said antigen of interest is entrapped by said protein matrix, and said composition is comprised of protein matrix particles having a mean particle size greater than 100 nm diameter.

2. The composition of claim 1, wherein said composition comprises protein matrix particles having a mean particle size diameter of greater than 120 nm, greater than 170 nm, greater than 200 nm, greater than 500 nm, greater than 1000 nm, greater than 2000 nm, or larger.

3. The composition of claim 1, wherein said composition comprises protein matrix particles having a mean particle size diameter of 100 nm to 2000 nm.

4. The composition of claim 1, wherein said composition comprises protein matrix particles having a particle size range from 100 to 2000 nm diameter.

5. The composition of claim 1, wherein the molar ratio of the antigen to the carrier protein is between 1 to 10 and 10 to 1.

6. The composition of claim 1, wherein said antigen of interest comprises two or more antigens.

7. The composition of claim 1, wherein said antigen of interest is a polysaccharide.

8. The composition of claim 7, wherein the polysaccharide is selected from the group consisting of a Streptococcus pneumoniae polysaccharide, Francisella tularensis polysaccharide, Bacillus anthracis polysaccharide, Haemophilus influenzae polysaccharide, Salmonella typhi polysaccharide, Citrobacter freundii polysacchardie, Salmonella species polysaccharide, Shigella polysaccharide, or Neisseria meningitidis polysaccharide.

9. The composition of claim 8, wherein said Streptococcus pneumoniae polysaccharide is selected from the group consisting of capsular type 3, 4, 6B, 7A, 7B, 7C, 7F, 9A, 9L, 9N, 9V, 12A, 12B, 12F, 14, 15A, 15B, 15C, 15F, 17, 18B, 18C, 19F, 23F, 25A, 25F, 33F, 35, 37, 38, 44, or 46.

10. The composition of claim 1, wherein the carrier protein is selected from the group consisting of diphtheria toxoid, CRM197, tetanus toxoid, Pseudomonas aeruginosa exotoxin A or a mutant thereof, cholera toxin B subunit, tetanus toxin fragment C, bacterial flagellin, pneumolysin, an outer membrane protein of Neisseria menningitidis, Pseudomonas aeruginosa Hcp1 protein, Escherichia coli heat labile enterotoxin, shiga-like toxin, human LTB protein, listeriolysin O, a protein extract from whole bacterial cells, the dominant negative inhibitor (DNI) mutant of the protective antigen of Bacillus anthracis, or Escherichia coli beta-galactosidase.

11. A method of making an immunogenic composition comprising (i) mixing an antigen of interest with a carrier protein to form a mixture and (ii) crosslinking said carrier protein to form a carrier protein matrix entrapping said antigen of interest, wherein no more than 50% of said antigen of interest is crosslinked to said carrier protein in said composition, and (iii) eliminating from the resulting composition protein matrix particles having a mean particle size diameter of less than 100 nm.

12. The method of claim 11, wherein, in step (iii) protein matrix particles having a mean particle size diameter of greater than 120 nm, greater than 170 nm, greater than 200 nm, greater than 500 nm, greater than 1000 nm, or greater than 2000 nm are selected.

13. The method of claim 12, wherein the protein matrix particles selected have a mean particle size diameter in the range of from 100 nm to 2000 nm.

14. The method of claim 12, wherein the protein matrix particles selected have a mean particle size diameter in the range of from 200 nm to 1000 nm.

15. The method of claim 12, wherein the protein matrix particles selected have a mean particle size diameter in the range of from 120 nm to 200 nm.

16. A method of making a protein matrix vaccine composition comprising (i) mixing an antigen of interest with a carrier protein and (ii) initiating a crosslinking reaction with a crosslinking agent that crosslinks functional groups on said carrier protein, and (iii) selecting from said reaction mixture protein matrix particles having a mean particle size diameter of greater than 100 nm.

17. The method of claim 16, wherein the protein matrix particles selected have a mean particle size diameter of greater than 120 nm, greater than 170 nm, greater than 200 nm, greater than 500 nm, greater than 1000 nm, or greater than 2000 nm.

18. The method of claim 16, wherein the protein matrix particles selected have a mean particle size diameter in the range of from 100 nm to 2000 nm.

19. The method of claim 17, wherein the protein matrix particles selected have a mean particle size diameter in the range of from 200 nm to 1000 nm.

20. A method of vaccinating a subject against an infectious agent, said method comprising administering a composition according to claim 1 to a subject in an amount sufficient to elicit an immune response.
